(19) United States
(12) Patent Application Publication
(10) Pub. No.: US 2020/0078574 A1
(43) Pub. Date: Mar. 12, 2020

(54) METHOD FOR MANUFACTURING MINUTE HOLLOW PROTRUDING TOOL, AND MINUTE HOLLOW PROTRUDING TOOL

(71) Applicant: KAO CORPORATION, Tokyo (JP)

(72) Inventors: Takatoshi NIITSU, Utsunomiya-shi, Tochigi (JP); Satoshi UENO, Utsunomiya-shi, Tochigi (JP)

(73) Assignee: KAO CORPORATION, Tokyo (JP)

(21) Appl. No.: 16/089,750

(22) PCT Filed: Mar. 30, 2017
(86) PCT No.: PCT/JP2017/013141

§ 371 Date: Sep. 28, 2018

(30) Foreign Application Priority Data
Mar. 31, 2016 (JP) 2016-071374

(51) Int. Cl. A61M 37/00; A61K 9/00

(52) U.S. Cl. CPC A61M 37/0015; A61K 9/0021; A61M 2037/0046; B29L 2031/7544; A61M 2037/003; A61M 2037/0053; A61M 2037/0061; A61M 2210/04

(57) ABSTRACT

A method for manufacturing a fine hollow protruding tool (1) having an opening portion (3h) of the present invention includes: a protrusion forming step of contacting a protrusion-forming projecting mold part (11A) including a heating means with first face (2D) of a base material sheet (2A), and inserting the protrusion-forming projecting mold part (11A) while softening, by heat, a contact point (TP) to form a fine hollow protrusion (3) which projects from the second face (2U) and which does not penetrate the projecting base material sheet (2A); a cooling step of cooling of cooling the fine hollow protrusion (3) in a state where the protrusion-forming projecting mold part (11A) is inserted therein; a release step of, after the cooling step, withdrawing the protrusion-forming projecting mold part (11A) to form a fine hollow protrusion (3) having a hollow interior; and an opening portion forming step of forming an opening portion (3h) which penetrates an interior portion of the fine hollow protrusion (3), at a position offset from a tip portion of the formed fine hollow protrusion (3).

### TECHNICAL FIELD

The present invention relates to a method for manufacturing a fine hollow protruding tool having opening portions. Furthermore, the present invention relates to a fine hollow protruding tool having opening portions.

### BACKGROUND ART

Recently, the delivery of agents using microneedles has been gaining attention in the medical field and the beauty field. Microneedles can achieve performance, without inducing pain, same as to deliver agents using syringes to pierce a shallow layer of the skin. Among microneedles, in particular, hollow microneedles with opening portions are effective because they can increase the number of choices of agents to be provided inside the microneedles. However, particularly when used in the medical or beauty field, hollow microneedles with opening portions need to have a high level of precision in their shape, and to have stability that enables to stably deliver agents through the opening portions into the skin.

Hollow microneedles with opening portions can be manufactured, for example, using manufacturing methods described in Patent Literatures 1 to 3. Patent Literature 1 describes a method using a mold part including a plurality of depressions formed in advance and a mold part including a plurality of projections formed in advance, inserting the projections into the depressions respectively, and manufacturing a hollow microneedle array through injection molding.

Furthermore, Patent Literature 2 describes a method forming opening portions of fine microneedles, which is reproduced on a substrate through heat imprinting, by using short pulse laser light, and manufacturing fine microneedles with fine opening portions.

Furthermore, Patent Literature 3 describes a method forming solid microneedles through heat cycle injection molding, forming channel holes through laser drilling, and manufacturing hollow microneedles having average channel holes with a length of less than 1 mm and a cross-sectional area of 20 to 50 μm².

### CITATION LIST

### Patent Literature

Patent Literature 1: US 2012041337(A1)

Patent Literature 2: JP 2011-72695A

Patent Literature 3: US 2011213335(A1)

### SUMMARY OF INVENTION

The present invention is directed to a method for manufacturing a fine hollow protruding tool. The method of the invention includes: a protrusion forming step of contacting a protrusion-forming projecting mold part including a heating means with a first face of a base material sheet containing a thermoplastic resin, and inserting the protrusion-forming projecting mold part into the base material sheet toward a second face of the base material sheet while softening, by heat, a contact portion of the base material sheet which contacts with the protrusion-forming projecting mold part to form a fine hollow protrusion which projects from the second face of the base material sheet and which does not penetrate the projecting base material sheet; and a cooling step of cooling the fine hollow protrusion in a state where the protrusion-forming projecting mold part is inserted in the fine hollow protrusion. The invention further includes: a release step, as a step following the cooling step, of withdrawing the protrusion-forming projecting mold part from the interior of the fine hollow protrusion to form the fine hollow protrusion having a hollow interior; and an opening portion forming step of forming an opening portion, which penetrates an interior portion of the fine hollow protrusion, at a position offset from a center of a tip portion of the formed fine hollow protrusion.

Furthermore, the present invention is directed to a fine hollow protruding tool including a fine hollow protrusion having an opening portion. The opening portion is arranged at a position offset from a center of a tip portion of the fine hollow protrusion, and penetrates a hollow interior portion of the fine hollow protrusion. The fine hollow protrusion includes a rising portion rising in the shape of a convex curve toward the interior of the fine hollow protrusion, at a peripheral edge of the opening portion.

### BRIEF DESCRIPTION OF DRAWINGS

FIG. 1 is a schematic perspective view of an example of a fine hollow protruding tool, in which fine hollow protrusions having opening portions are arranged in an array, manufactured using a method for manufacturing a fine hollow protruding tool having opening portions of the present invention.

FIG. 2 is a perspective view of the fine hollow protruding tool focusing on one fine hollow protrusion shown in FIG. 1.

FIG. 3 is a cross-sectional view taken along line shown in FIG. 2.

FIG. 4 is a view showing the overall configuration according to an embodiment of a manufacturing apparatus for manufacturing the fine hollow protruding tool shown in FIG. 1.

FIG. 5 is an explanatory view showing a method for measuring the tip diameter and the tip angle of a projection of a projecting mold part.

FIGS. 6(a) to 6(f) are views illustrating steps for manufacturing a fine hollow protruding tool having opening portions using the manufacturing apparatus shown in FIG. 4.

FIG. 7 is a view illustrating another manufacturing method for manufacturing the fine hollow protruding tool shown in FIG. 1.

FIG. 8 is a view illustrating another manufacturing method for manufacturing the fine hollow protruding tool shown in FIG. 1.

FIGS. 9(a) and 9(b) are views illustrating a manufacturing method for manufacturing a fine hollow protruding tool in a form different from that shown in FIG. 1.

FIG. 10 is a view illustrating another manufacturing method for manufacturing a fine hollow protruding tool in a form different from that shown in FIG. 1.

FIG. 11 is a view illustrating another manufacturing method for manufacturing a fine hollow protruding tool in a form different from that shown in FIG. 1.

FIGS. 12A-B are photographs of the manufactured fine hollow protruding tool of Example 1 observed using a microscope.

FIG. 13 is a photograph of the manufactured fine hollow protruding tool of Comparative Example 1 observed using a microscope.

### DESCRIPTION OF EMBODIMENT

According to the manufacturing method described in Patent Literature 1, manufacture is performed using injection molding, and thus the temperature is likely to vary between a depressed mold part and a projecting mold part that are used, and the mold parts are likely to be deformed by wearing down. Thus, it is difficult to manufacture microneedles with a precise shape, which makes it difficult to stably deliver agents through the opening portions into the skin.

Furthermore, according to the manufacturing methods described in Patent Literatures 2 and 3, after microneedles have been formed in a preceding step, opening portions are formed using laser light in subsequent processing. Thus the microneedles formed by mold parts in the preceding step have to be released from the mold parts and the positioning is reset. Accordingly, it is difficult to precisely perform irradiation with laser light, which makes it difficult to manufacture microneedles with a precise shape having opening portions.

The present invention relates to a method for manufacturing a fine hollow protruding tool having opening portions that can solve the above-mentioned problems in the related art. Furthermore, the present invention relates to a fine hollow protruding tool having opening portions that can solve the above-mentioned problems in the related art.

Hereinafter, the present invention will be described with reference to the drawings based on a preferable embodiment.

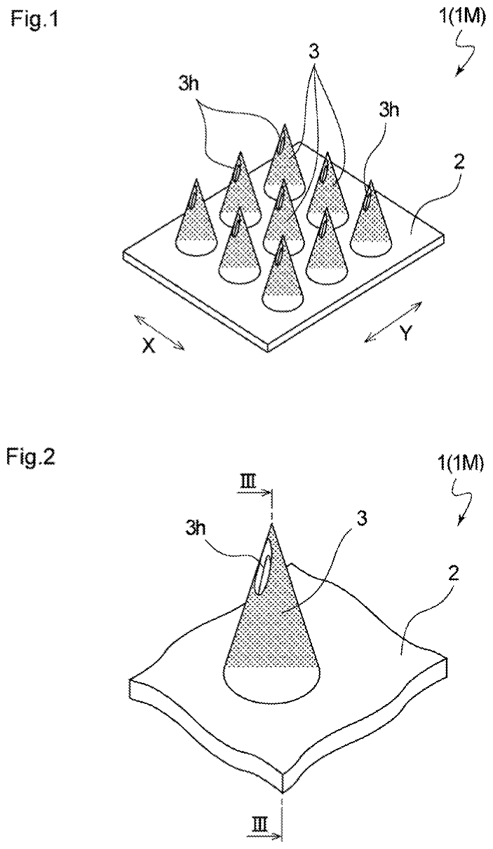

FIG. 1 shows a perspective view of a microneedle array 1M as a fine hollow protruding tool 1 of a preferable embodiment of the fine hollow protruding tool of the present invention. The microneedle array 1M of this embodiment includes fine hollow protrusions 3 having opening portions 3h. The microneedle array 1M has a form in which fine hollow protrusions 3 project from a basal member 2, the fine hollow protrusions 3 each having an opening portion 3h on the tip side, and an interior in which an interior space linked to the opening portion 3h is formed. The microneedle array 1M of this embodiment includes a sheet-like basal member 2 and a plurality of fine hollow protrusions 3.

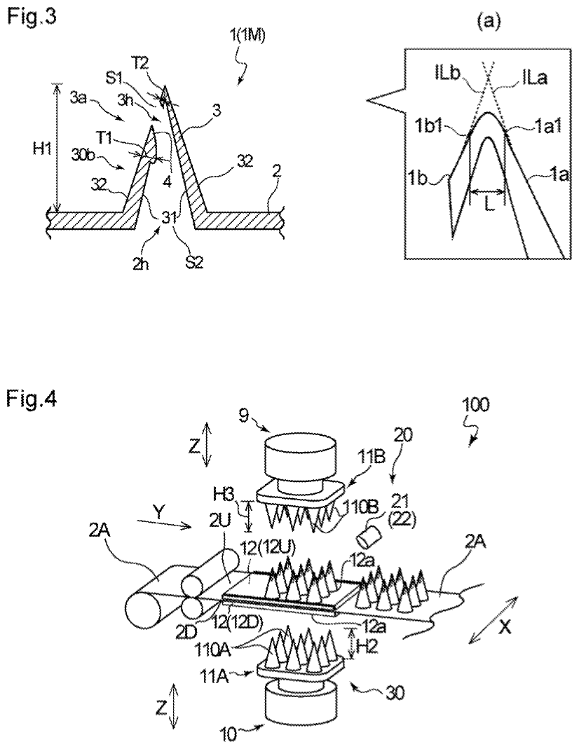

There is no particular limitation on the number of fine hollow protrusions 3, the arrangement of the fine hollow protrusions 3, and the shape of the fine hollow protrusions 3. In the microneedle array 1M of this embodiment, nine truncated conical fine hollow protrusions 3 are arranged in an array on the upper face of the sheet-like basal member 2. The nine fine hollow protrusions 3 arranged in an array are arranged in three rows along a Y direction, which is the direction in which a later-described base material sheet 2A is transported (the longitudinal direction of the base material sheet 2A), and in three columns along an X direction, which is the direction orthogonal to the transporting direction and which is the lateral direction of the base material sheet 2A that is being transported. Note that FIG. 2 is a perspective view of the microneedle array 1M focusing on one fine hollow protrusion 3 from among the fine hollow protrusions 3 arranged in an array included in the microneedle array 1M, and FIG. 3 is a cross-sectional view taken along line shown in FIG. 2.

The microneedle array 1M has the opening portions 3h as shown in FIG. 2. Furthermore, the microneedle array 1M is such that spaces extending from the basal member 2 to the opening portions 3h are formed in the interiors of each of the fine hollow protrusions 3, as shown in FIG. 3. In the microneedle array 1M of this embodiment, the opening portions 3h are arranged at positions offset from the centers of the tip portions of the fine hollow protrusions 3, and penetrate the hollow interior portions of the fine hollow protrusions 3. If the opening portions 3h are arranged at positions offset from the centers of the tip portions of the fine hollow protrusions 3 in this manner, when using the fine hollow protrusions 3 of the microneedle array 1M to pierce the skin, the opening portions 3h are unlikely to be crushed, and thus it is possible to stably deliver agents through the opening portions 3h into the skin. In the microneedle array 1M, the interior spaces of the fine hollow protrusions 3 are formed in a shape that conforms to the outer shape of the fine hollow protrusions 3, and, in this embodiment, they are formed in a conical shape, which is a shape that conforms to the outer shape of the conical fine hollow protrusions 3. Note that, although the fine hollow protrusions 3 are in a conical shape in this embodiment, they may be in a pyramidal shape or the like instead of a conical shape.

In the microneedle array 1M of this embodiment, each fine hollow protrusion 3 has a rising portion 4 rising in the shape of a convex curve toward the interior of the fine hollow protrusion 3, at the peripheral edge of the opening portion 3h. Preferably, in a vertical cross-section passing through the apex of the fine hollow protrusion 3 and the center of the opening portion 3h (see FIG. 3), the fine hollow protrusion 3 has the rising portion 4 at least on the lower side of the peripheral edge of the opening portion 3h, in one wall portion 3a on the side having the opening portion 3h. As shown in FIG. 3, the rising portion 4 rises inward from the peripheral edge of the opening portion 3h, in the shape of a convex curve toward the interior of the fine hollow protrusion 3. As shown in FIG. 3, each rising portion 4 in the microneedle array 1M is such that a wall thickness T1 on the lower side of the peripheral edge of the opening portion 3h (a gap between the apex portion of the rising portion 4 on the lower side of the peripheral edge of the opening portion 3h and an outer wall 32) is thicker than a wall thickness T2 on the upper side of the peripheral edge of the opening portion 3h (a gap between the apex portion of the rising portion 4 on the upper side of the peripheral edge of the opening portion 3h and the outer wall 32). Furthermore, in the microneedle array 1M of this embodiment, as shown in FIG. 3, an outer wall 32 of a lower wall portion 30b on the lower side forming the one wall portion 3a on the side having the opening portion 3h is formed in the shape of a straight line, and the inner wall 31 of the lower wall portion 30b excluding the rising portion 4 is also formed in the shape of a straight line. If the peripheral edge of the opening portion 3h has the rising portion 4 in this manner, when using the fine hollow protrusions 3 of the microneedle array 1M to pierce the skin, the opening portions 3h are less likely to be crushed. Furthermore, since the rising portion 4 rises inward, when using the fine hollow protrusion 3 to pierce the skin, piercing can be smoothly performed. Accordingly, it is possible to stably deliver agents through the opening portion 3h into the skin.

Each fine hollow protrusion 3 in the microneedle array 1M is inserted such that its tip reaches the stratum corneum, which is the outermost layer, or the dermis, which is a deeper layer, and thus a projecting height H1 thereof is preferably 0.01 mm or greater, and more preferably 0.02 mm or greater, is preferably 10 mm or less, and more preferably 5 mm or less, and, specifically, is preferably from 0.01 to 10 mm, and more preferably from 0.02 to 5 mm.

The tips of the fine hollow protrusions 3 in the microneedle array 1M each have a tip diameter L (the gap between the outer walls 32 at the tip) that is preferably 1 μm or greater, and more preferably 5 μm or greater, is preferably 500 μm or less, and more preferably 300 μm or less, and, specifically, is preferably from 1 to 500 μm, and more preferably from 5 to 300 μm. The tip diameter L of the fine hollow protruding tool 1 is the length at a position where the length is longest at the tip of a fine hollow protrusion 3. If the tip diameter L is within the above-described range, there is almost no pain when the microneedle array 1M is inserted into the skin. The tip diameter L is measured as follows.

### Measurement of Tip Diameter of Fine Hollow Protrusions 3 in Microneedle Array 1M

The tip portion of the fine hollow protrusion 3 is observed in a state of being enlarged at a predetermined magnification as shown in FIG. 3(a) using a scanning electron microscope (SEM) or a microscope.

Next, as shown in FIG. 3(a), an imaginary straight line ILa is extended along the straight-line portion of one lateral side 1a of two lateral sides 1a and 1b defining the outer walls 32, and an imaginary straight line ILb is extended along the straight-line portion of the other lateral side 1b. Next, the point where the lateral side 1a separates from the imaginary straight line ILa on the tip side is defined as a first tip point 1a1, and the point where the other lateral side 1b separates from the imaginary straight line ILb is defined as a second tip point 1b1. A length L of a straight line that connects the first tip point 1a1 and the second tip point 1b1 defined as above is measured using a scanning electron microscope (SEM) or a microscope, and the measured length of the straight line is defined as the tip diameter of the fine hollow protrusion 3.

As shown in FIG. 3, the fine hollow protruding tool 1 includes an opening portion 3h arranged at a position offset from the center of the tip portion of each fine hollow protrusion 3, and a basal-side opening portion 2h arranged in the lower face of the basal member 2 corresponding to the fine hollow protrusion 3.

The opening portion 3h has an opening area 51 that is preferably 0.7 μm² or greater, and more preferably 20 μm² or greater, is preferably 200000 μm² or less, and more preferably 70000 μm² or less, and, specifically, is preferably from 0.7 to 200000 μm², and more preferably from 20 to 70000 μm².

The basal-side opening portion 2h has an opening area S2 that is preferably 0.007 mm² or greater, and more preferably 0.03 mm² or greater, is preferably 20 mm² or less, and more preferably 7 mm² or less, and, specifically, is preferably from 0.007 to 20 mm², and more preferably from 0.03 to 7 mm².

The nine fine hollow protrusions 3 arranged in an array on the upper face of the sheet-like basal member 2 are preferably such that the center-to-center distance in the longitudinal direction (Y direction) is uniform and the center-to-center distance in the lateral direction (X direction) is uniform, and, preferably, the center-to-center distance in the longitudinal direction (Y direction) is the same as the center-to-center distance in the lateral direction (X direction). Preferably, the center-to-center distance in the longitudinal direction (Y direction) between the fine hollow protrusions 3 is preferably 0.01 mm or greater, and more preferably 0.05 mm or greater, is preferably 10 mm or less, and more preferably 5 mm or less, and, specifically, is preferably from 0.01 to 10 mm, and more preferably from 0.05 to 5 mm. Furthermore, the center-to-center distance in the lateral direction (X direction) between the fine hollow protrusions 3 is preferably 0.01 mm or greater, and more preferably 0.05 mm or greater, is preferably 10 mm or less, and more preferably 5 mm or less, and, specifically, is preferably from 0.01 to 10 mm, and more preferably from 0.05 to 5 mm.

Next, a method for manufacturing the fine hollow protruding tool of the present invention will be described with reference to FIGS. 4 to 6, using a method for manufacturing the microneedle array 1M as the fine hollow protruding tool 1 described above as an example. FIG. 4 shows the overall configuration of a manufacturing apparatus 100 according to an embodiment used for implementing the manufacturing method of this embodiment. Note that, although the fine hollow protrusions 3 in the microneedle array 1M are very small as described above, the fine hollow protrusions 3 in the microneedle array 1M are illustrated very large in FIG. 4.

The manufacturing apparatus 100 of this embodiment shown in FIG. 4 includes a protrusion forming section 10 for forming fine hollow protrusions 3 on the base material sheet 2A, a cooling section 20, a release section 30 for withdrawing a later-described protrusion-forming projecting mold part 11A, and an opening portion forming section 9 for forming opening portions 3h that penetrate the hollow interior portions of the fine hollow protrusions 3.

In the description below, the direction in which the base material sheet 2A is transported (the longitudinal direction of the base material sheet 2A) is referred to as a Y direction, the direction that is orthogonal to the transporting direction, which is the lateral direction of the base material sheet 2A that is being transported, is referred to as an X direction, and the thickness direction of the base material sheet 2A that is being transported is referred to as a Z direction.

The base material sheet 2A is a sheet that is formed into the basal member 2 included in the microneedle array 1M that is to be manufactured, and contains a thermoplastic resin. The base material sheet 2A is preferably a sheet mainly made of thermoplastic resin, that is, containing 50% by mass or greater of thermoplastic resin, and more preferably a sheet containing 90% by mass or greater of thermoplastic resin. Examples of the thermoplastic resin include poly-fatty acid esters, polycarbonate, polypropylene, polyethylene, polyester, polyamide, polyamide imide, polyether ether ketone, polyetherimide, polystyrene, polyethylene terephthalate, polyvinyl chloride, nylon resin, acrylic resin, and combinations thereof. From the viewpoint of biodegradability, poly-fatty acid esters are preferably used. Specific examples of poly-fatty acid esters include polylactic acid, polyglycolic acid, and combinations thereof. Note that the base material sheet 2A may be formed of a mixture including, for example, hyaluronic acid, collagen, starch, cellulose, etc., in addition to thermoplastic resin. The thickness of the base material sheet 2A is similar to the thickness T2 of the basal member 2 included in the microneedle array 1M that is to be manufactured.

As shown in FIG. 4, the protrusion forming section 10 includes the protrusion-forming projecting mold part 11A including a heating means (not shown). The protrusion-forming projecting mold part 11A has projections 110A corresponding to the number and arrangement of fine hollow protrusions 3 of the microneedle array 1M that is to be manufactured, and corresponding substantially to the outer shape of each fine hollow protrusion 3. In the manufacturing apparatus 100 of this embodiment, nine conical projections 110A are provided corresponding to the nine truncated conical fine hollow protrusions 3.

In the manufacturing apparatus 100 of this embodiment, as shown in FIG. 4, nine conical projections 110A with sharp tips are arranged in the protrusion-forming projecting mold part 11A such that the tips face upward, and the protrusion-forming projecting mold part 11A is movable at least vertically in the thickness direction (Z direction). In the manufacturing apparatus 100 of this embodiment, the protrusion-forming projecting mold part 11A can be moved vertically in the thickness direction (Z direction) by an electric actuator (not shown).

As shown in FIG. 4, the opening portion forming section 9 includes an opening-forming projecting mold part 11B including a heating means (not shown). In the manufacturing apparatus 100 of this embodiment, as shown in FIG. 4, the protrusion-forming projecting mold part 11A included in the protrusion forming section 10 is different from the opening-forming projecting mold part 11B included in the opening portion forming section 9. The opening-forming projecting mold part 11B has projections 110B corresponding to the number of fine hollow protrusions 3 of the microneedle array 1M that is to be manufactured, and, in the manufacturing apparatus 100 of this embodiment, nine conical projections 110B are provided corresponding to the nine truncated conical fine hollow protrusions 3.

In the manufacturing apparatus 100 of this embodiment, as shown in FIG. 4, nine conical projections 110B with sharp tips are arranged in the opening-forming projecting mold part 11B such that the tips face downward, and the opening-forming projecting mold part 11B is movable at least vertically in the thickness direction (Z direction). In the manufacturing apparatus 100 of this embodiment, the opening-forming projecting mold part 11B can be moved vertically in the thickness direction (Z direction) by the electric actuator (not shown).

In the manufacturing apparatus 100 of this embodiment, as shown in FIG. 4, the projections 110A of the protrusion-forming projecting mold part 11A included in the protrusion forming section 10 are arranged such that the tips face upward, the projections 110B of the opening-forming projecting mold part 11B included in the opening portion forming section 9 are arranged such that the tips face downward, and the projecting mold parts 11A and 11B are movable vertically in the thickness direction (Z direction). In this manner, in the manufacturing apparatus 100 of this embodiment, an insertion angle θ1 of the protrusion-forming projecting mold part 11A with respect to the base material sheet 2A is different from an insertion angle θ2 of the opening-forming projecting mold part 11B with respect to the base material sheet 2A, and a difference therebetween is 180 degrees. Accordingly, the manufacturing apparatus 100 of this embodiment is configured such that the protrusion-forming projecting mold part 11A is brought into contact with a first face 2D (the lower face) of the base material sheet 2A, and the opening-forming projecting mold part 11B is brought into contact with a second face 2U (the upper face) of the base material sheet 2A.

Note that, in this specification, the protrusion-forming projecting mold part 11A and the opening-forming projecting mold part 11B (hereinafter, they may be collectively referred to as projecting mold parts 11A and 11B, or as projecting mold parts 11 with no distinction) are members including the projections 110A and 110B respectively corresponding to the projecting mold parts 11A and 11B, the projections 110A and 110B being portions that are inserted into the base material sheet 2A. In the manufacturing apparatus 100 of this embodiment, the projecting mold parts 11A and 11B are arranged on a disk-like stage portion. Note that the configuration of the projecting mold parts 11A and 11B is not limited to this, and they each may be a projecting mold part including only the projections 110A or 110B, or may be projecting mold parts 11A or 11B in which the plurality of projections 110A or 110B are arranged on a table-like support.

In the manufacturing apparatus 100 of this embodiment, the operation of each of the projecting mold parts 11A and 11B (electric actuator) is controlled by a control means (not shown) included in the manufacturing apparatus 100 of this embodiment. Note that it is preferable that the heating means (not shown) of each of the projecting mold parts 11A and 11B is operated from immediately before the protrusion-forming projecting mold part 11A comes into contact with a target to immediately before the procedure reaches a later-described cooling step.

The operation of the projecting mold parts 11A and 11B, and heating conditions of the heating means (not shown) included in the projecting mold parts 11A and 11B, such as the operation of the heating means (not shown) of the projecting mold parts 11A and 11B, are controlled by a control means (not shown) included in the manufacturing apparatus 100 of this embodiment.

In this embodiment, the condition of the amount of processing heat in the protrusion forming section 10 is different from the condition of the amount of processing heat in the opening portion forming section 9. In the manufacturing apparatus 100, the protrusion-forming projecting mold part 11A that is used in the protrusion forming section 10 is different from the opening-forming projecting mold part 11B that is used in the opening portion forming section 9, and the amount of processing heat that is applied from the protrusion-forming projecting mold part 11A to the base material sheet 2A is larger than the amount of processing heat that is applied from the opening-forming projecting mold part 11B to the fine hollow protrusions 3. Here, the amount of processing heat that is applied to the base material sheet 2A refers to the amount of heat that is applied to the base material sheet 2A per unit insertion height. The amount of processing heat that is applied to the fine hollow protrusions 3 refers to the amount of heat that is applied to the fine hollow protrusions 3 per unit insertion height, as in the amount of heat that is applied to the base material sheet 2A. Specifically, the condition that makes the amount of processing heat that is applied from the protrusion-forming projecting mold part 11A to the base material sheet 2A in the protrusion forming section 10 larger than the amount of processing heat that is applied from the opening-forming projecting mold part 11B to the fine hollow protrusions 3 in the opening portion forming section 9 refers to satisfying at least one of: (Condition a) the insertion speed of the protrusion-forming projecting mold part 11A into the base material sheet 2A and the insertion speed of the opening-forming projecting mold part 11B into the fine hollow protrusions 3 are such that the insertion speed of the protrusion forming section 10 is slower than the insertion speed of the opening portion forming section 9; (Condition b) in the case where the heating means (not shown) of each of the projecting mold parts 11A and 11B is an ultrasonic vibration device, the frequency of ultrasonic waves in the protrusion-forming projecting mold part 11A is higher than the frequency of ultrasonic waves in the opening-forming projecting mold part 11B; (Condition c) in the case where the heating means (not shown) of each of the projecting mold parts 11A and 11B is an ultrasonic vibration device, the amplitude of ultrasonic waves in the protrusion-forming projecting mold part 11A is larger than the amplitude of ultrasonic waves in the opening-forming projecting mold part 11B; and (Condition d) in the case where the heating means (not shown) of each of the projecting mold parts 11A and 11B is a heater device, the heater temperature of the protrusion-forming projecting mold part 11A is higher than the heater temperature of the opening-forming projecting mold part 11B.

Note that, in the manufacturing apparatus that is used in the method for manufacturing the fine hollow protruding tool of the present invention, no heating means is provided other than the heating means (not shown) of the projecting mold parts 11A and 11B. In this specification, “no heating means is provided other than the heating means of the projecting mold parts 11A and 11B” refers to not only cases in which other heating means are completely excluded but also cases in which a means for heating the base material sheet 2A to a temperature below its softening temperature, and preferably below its glass transition temperature is provided. Specifically, additional heating means which heat the base material sheet 2A to a temperature below the softening temperature may be provided, as long as the temperature of the base material sheet 2A reached due to heating by the heating means of each of the projecting mold parts 11A and 11B is the same as or greater than the softening temperature of the base material sheet 2A. Furthermore, additional heating means which heat the base material sheet 2A to a temperature below the glass transition temperature may be provided, as long as the temperature of the base material sheet 2A reached due to heating by the heating means of each of the projecting mold parts 11A and 11B is the same as or greater than the glass transition temperature and that is below the softening temperature. Note that it is preferable that no heating means other than the heating means provided at the projecting mold parts 11A and 11B is provided at all.

In the manufacturing apparatus 100 of this embodiment, the heating means (not shown) of each of the projecting mold parts 11A and 11B is an ultrasonic vibration device.

The projections 110A of the protrusion-forming projecting mold part 11A have an outer shape that is sharper than the outer shape of the fine hollow protrusions 3 included in the microneedle array 1M. The projections 110A of the protrusion-forming projecting mold part 11A each have a height H2 (see FIG. 4) that is higher than the height H1 of the microneedle array 1M that is to be manufactured, and it is preferably 0.01 mm or greater, and more preferably 0.02 mm or greater, is preferably 30 mm or less, and more preferably 20 mm or less, and, specifically, is preferably from 0.01 to 30 mm, and more preferably from 0.02 to 20 mm.

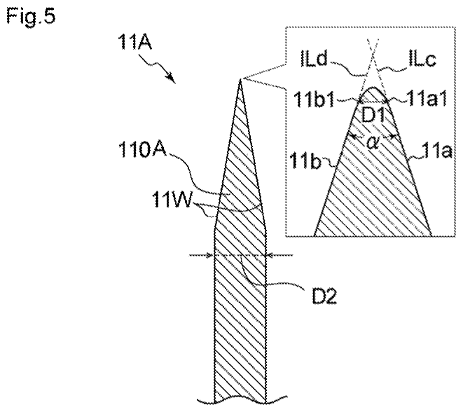

The projections 110A of the protrusion-forming projecting mold part 11A each have a tip diameter D1 (see FIG. 5) that is preferably 0.001 mm or greater, and more preferably 0.005 mm or greater, is preferably 1 mm or less, and more preferably 0.5 mm or less, and, specifically, is preferably from 0.001 to 1 mm, and more preferably from 0.005 to 0.5 mm. The tip diameter D1 of the projections 110A of the protrusion-forming projecting mold part 11A is measured as follows.

The projections 110A of the protrusion-forming projecting mold part 11A each have a base diameter D2 (see FIG. 5) that is preferably 0.1 mm or greater, and more preferably 0.2 mm or greater, is preferably 5 mm or less, and more preferably 3 mm or less, and, specifically, is preferably from 0.1 to 5 mm, and more preferably from 0.2 to 3 mm.

The projections 110A of the protrusion-forming projecting mold part 11A each have a tip angle α (see FIG. 5) that is preferably 1 degree or greater, and more preferably 5 degrees or greater, in order to facilitate making the projections 110A sufficiently strong. Furthermore, in order to obtain fine hollow protrusions 3 with an appropriate angle, the tip angle a is preferably 60 degrees or less, and more preferably 45 degrees or less, and, specifically, is preferably from 1 to 60 degrees, and more preferably from 5 to 45 degrees. The tip angle α of the projections 110A of the protrusion-forming projecting mold part 11A is measured as follows.

### Measurement of Tip Diameter of Projections 110A of Protrusion-Forming Projecting Mold Part 11A

The tip portion of the projection 110A of the protrusion-forming projecting mold part 11A is observed in a state of being enlarged at a predetermined magnification using a scanning electron microscope (SEM) or a microscope. Next, as shown in FIG. 5, an imaginary straight line ILc is extended along the straight-line portion of one lateral side 11a of two lateral sides 11a and 11b, and an imaginary straight line ILd is extended along the straight-line portion the other lateral side 11b. Then, a point where the lateral side 11a separates from the imaginary straight line ILc on the tip side is defined as a first tip point 11a1, and a point where the other lateral side 11b separates from the imaginary straight line ILd is defined as a second tip point 11b1. A length D1 of a straight line that connects the first tip point 11a1 and the second tip point 11b1 defined as above is measured using a scanning electron microscope (SEM), and the measured length of the straight line is defined as the tip diameter of the projection 110A.

### Measurement of Tip Angle a of Projections 110A of Protrusion-Forming Projecting Mold Part 11A

The tip portion of the projection 110A of the protrusion-forming projecting mold part 11A is observed in a state of being enlarged at a predetermined magnification using a scanning electron microscope (SEM) or a microscope. Next, as shown in FIG. 5, an imaginary straight line ILc is extended along the straight-line portion of one lateral side 11a of two lateral sides 11a and 11b, and an imaginary straight line ILd is extended along the straight-line portion the other lateral side 11b. An angle formed by the imaginary straight line ILc and the imaginary straight line ILd is measured using a scanning electron microscope (SEM), and the measured angle is defined as the tip angle a of the projection 110A of the protrusion-forming projecting mold part 11A.

The projections 110B of the opening-forming projecting mold part 11B may have the same outer shape as the projections 110A of the protrusion-forming projecting mold part 11A that is used in the protrusion forming section 10, but may have an outer shape different therefrom, in order to form the opening portions 3h at positions offset from the centers of the tip portions of the fine hollow protrusions 3.

The projections 110B of the opening-forming projecting mold part 11B each have a height H3 that is preferably 0.01 mm or greater, and more preferably 0.02 mm or greater, is preferably 30 mm or less, and more preferably 20 mm or less, and, specifically, is preferably from 0.01 to 30 mm, and more preferably from 0.02 to 20 mm.

The projections 110B of the opening-forming projecting mold part 11B may have the same tip diameter as the tip diameter D1 of the projections 110A of the protrusion-forming projecting mold part 11A (see FIG. 5), but it is preferably smaller than the tip diameter D1 of the projections 110A of the protrusion-forming projecting mold part 11A (see FIG. 5), in order to form the opening portions 3h at positions offset from the centers of the tip portions of the fine hollow protrusions 3. The projections 110B for opening-forming each have a tip diameter that is preferably 0.001 mm or greater, and more preferably 0.005 mm or greater, is preferably 1 mm or less, and more preferably 0.5 mm or less, and, specifically, is preferably from 0.001 to 1 mm, and more preferably from 0.005 to 0.5 mm. The tip diameter of the projections 110B is measured as in the tip diameter D1 of the projections 110A described above.

The projections 110B of the opening-forming projecting mold part 11B may have the same base diameter as the base diameter D2 of the projections 110A of the protrusion-forming projecting mold part 11A (see FIG. 5), but it is preferably smaller than the base diameter D2 of the projections 110A (see FIG. 5), in order to form the opening portions 3h at positions offset from the centers of the tip portions of the fine hollow protrusions 3. The projections 110B each have a base diameter that is preferably 0.1 mm or greater, and more preferably 0.2 mm or greater, is preferably 5 mm or less, and more preferably 3 mm or less, and, specifically, is preferably from 0.1 to 5 mm, and more preferably from 0.2 to 3 mm.

The projections 110B of the opening-forming projecting mold part 11B may have the same tip angle as the tip angle α of the projections 110A of the protrusion-forming projecting mold part 11A (see FIG. 5), but it is preferably smaller than the tip angle α of the projections 110A (see FIG. 5), in order to form the opening portions 3h at positions offset from the centers of the tip portions of the fine hollow protrusions 3. The projections 110B each have a tip angle that is preferably 1 degree or greater, and more preferably 5 degrees or greater, is preferably 60 degrees or less, and more preferably 45 degrees or less, and, specifically, is preferably from 1 to 60 degrees, and more preferably from 5 to 45 degrees. The tip angle of the projections 110B is measured as in the tip angle α of the projections 110A described above.

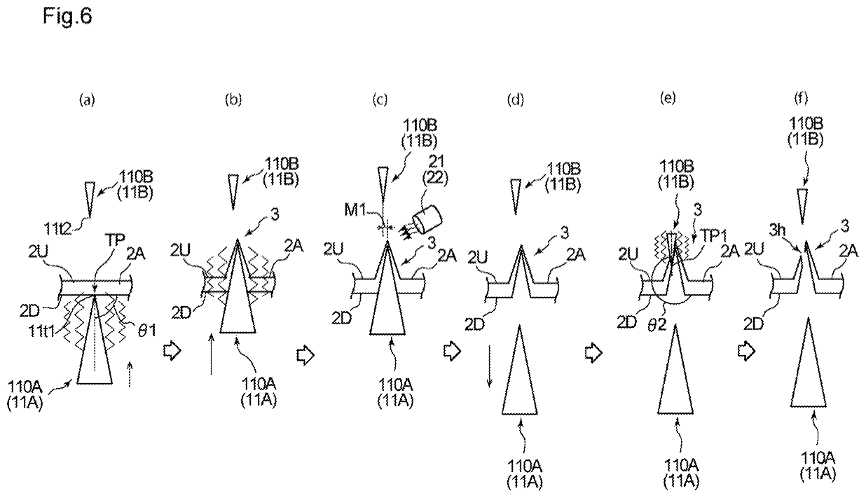

In the manufacturing apparatus 100 of this embodiment, as shown in FIG. 6, the protrusion-forming projecting mold part 11A and the opening-forming projecting mold part 11B are arranged such that a center 11t1 of the tip portion of each projection 110A of the protrusion-forming projecting mold part 11A is offset from a center 11t2 of the tip portion of each projection 110B of the opening-forming projecting mold part 11B. That is to say, the center of the tip portion of the fine hollow protrusion 3, which does not penetrate the projecting base material sheet 2A, formed by inserting the protrusion-forming projecting mold part 11A into the base material sheet 2A is offset from the center 11t2 of the tip portion of the projection 110B of the opening-forming projecting mold part 11B. In the manufacturing apparatus 100 of this embodiment, as shown in FIG. 6, the center 11t1 of the tip portion of the protrusion-forming projecting mold part 11A and the center 11t2 of the tip portion of the opening-forming projecting mold part 11B are offset from each other in the Y direction. Here, an offset amount M1 (see FIG. 6(c)) between the center 11t1 of the tip portion of the protrusion-forming projecting mold part 11A (the center of the tip portion of the fine hollow protrusion 3 which does not penetrate the base material sheet 2A that is projecting) and the center 11t2 of the tip portion of the opening-forming projecting mold part 11B is preferably within the half the base diameter D2 (see FIG. 5) of the projection 110A of the protrusion-forming projecting mold part 11A, and is preferably 0.001 mm or greater, and more preferably 0.005 mm or greater, is preferably 1.5 mm or less, and more preferably 1.0 mm or less, and, specifically, is preferably from 0.001 to 1.5 mm, and more preferably from 0.005 to 1.0 mm, in order to efficiently manufacture a microneedle array 1M including the fine hollow protrusions 3 having the opening portions 3h at positions offset from the centers of the tip portions.

The projecting mold parts 11A and 11B are made of a high-strength material that is hard to bend/break. Examples of the material for forming the projecting mold parts 11A and 11B include metals, such as steel, stainless steel, aluminum, an aluminum alloy, nickel, a nickel alloy, cobalt, a cobalt alloy, copper, a copper alloy, beryllium copper, and a beryllium copper alloy, and ceramics.

The protrusion forming section 10 in the manufacturing apparatus 100 of this embodiment includes a support 12 that supports the base material sheet 2A while the protrusion-forming projecting mold part 11A is inserted into the base material sheet 2A, as shown in FIG. 4. In this embodiment, an opening plate 12U having a plurality of openings 12a into which the projections 110 of the protrusion-forming projecting mold part 11A can be respectively inserted is used as the support 12. The opening plate 12U is arranged on the second face 2U side of the base material sheet 2A, and serves to make the base material sheet 2A less likely to warp/bend when the protrusion-forming projecting mold part 11A is inserted from the first face 2D. Accordingly, the opening plate 12U is arranged in a portion of the base material sheet 2A other than the region into which the protrusion-forming projecting mold part 11A is inserted. Meanwhile, the opening portion forming section 9 includes an opening plate 12D, serving as a support 12 that supports the base material sheet 2A when the opening-forming projecting mold part 11B is inserted into the fine hollow protrusions 3 of the base material sheet 2A. If the opening plate 12D is used, the base material sheet 2A can be kept stable when the protrusion-forming projecting mold part 11A is inserted and withdrawn.

In the manufacturing apparatus 100 of this embodiment, the opening plates 12U and 12D are arranged throughout the protrusion forming section 10, the cooling section 20, the release section 30, and the opening portion forming section 9. The opening plates 12U and 12D are constituted by plate-like members that extend parallel to the transporting direction (Y direction). The opening plates 12U and 12D support the base material sheet 2A at regions thereof other than the openings 12a.

Each of the opening plates 12U and 12D may be formed such that a single opening 12a has a greater opening area than the cross-sectional area of the projections 110A and 110B of the projecting mold parts 11A and 12B so that a plurality of projections 110A and 110B can be passed through a single opening, but, in the manufacturing apparatus 100 of this embodiment, as shown in FIG. 4, the opening plates 12U and 12D are formed such that one projection 110A and one projection 110B are passed through one opening 12a.

The opening plates 12U and 12D are movable in directions contacting to and separating from the base material sheet 2A. In the manufacturing apparatus 100 of this embodiment, the opening plates 12U and 12D can be moved vertically in the thickness direction (Z direction) by the electric actuator (not shown).

The operation of the opening plates 12U and 12D is controlled by a control means (not shown) included in the manufacturing apparatus 100 of this embodiment.

Although the opening plates 12U and 12D are movable in directions contacting to and separating from the base material sheet 2A in this embodiment, the opening plate 12D, which is one of the opening plates, does not have to be movable in directions contacting to and separating from the base material sheet 2A.

The material for forming the support 12 (the opening plates 12U and 12D) may be the same as the material for forming the projecting mold parts 11A and 11B, and may be formed of a synthetic resin, for example.

Furthermore, in the manufacturing apparatus 100 of this embodiment, as shown in FIG. 4, the cooling section 20 is provided subsequent to the protrusion forming section 10. As shown in FIG. 4, the cooling section 20 includes the cold air blowing device 21. In the manufacturing apparatus 100 of this embodiment, the cold air blowing device 21 is provided with an air vent 22 for blowing cold air onto the second face 2U side (the upper face side) of the base material sheet 2A, and the fine hollow protrusions 3 are cooled by cold air blown from the air vent 22. Note that the cold air blowing device may be configured so as to cover, in a hollow shape, the entirety of the second face 2U side (the upper face side) and the first face 2D side (the lower face side) of the continuous base material sheet 2A being transported, so that the continuous base material sheet 2A is transported along the transporting direction (Y direction) inside the cold air blowing device, and the air vent 22 for blowing cold air may be provided in the hollow. The cooling temperature and the cooling time of the cold air blowing device 21 are controlled by a control means (not shown) included in the manufacturing apparatus 100 of this embodiment.

Furthermore, in the manufacturing apparatus 100 of this embodiment, as shown in FIG. 4, the release section 30 is provided subsequent to the cooling section 20. In the release section 30, as described above, the protrusion-forming projecting mold part 11A can be moved downward in the thickness direction (Z direction) by the electric actuator (not shown).

The method for manufacturing the fine hollow protruding tool 1 (the microneedle array 1M) having the opening portions 3h of this embodiment includes a protrusion forming step of contacting the protrusion-forming projecting mold part 11A including the heating means with the first face 2D (the lower face) of the base material sheet 2A containing a thermoplastic resin, and inserting the projecting mold part into the base material sheet 2A toward the second face 2U (the upper face) of the base material sheet 2A while softening, by heat, a contact portion TP of the base material sheet 2A which contacts with the protrusion-forming projecting mold part 11A to form a fine hollow protrusions 3 which project from the second face 2U (the upper face) of the base material sheet 2A and which do not penetrate the projecting base material sheet 2A. Furthermore, this embodiment includes a cooling step of cooling the fine hollow protrusions 3 in a state where the protrusion-forming projecting mold part 11A is inserted in the fine hollow protrusions 3 as a step following the protrusion forming step. Furthermore, this embodiment includes a release step of withdrawing the protrusion-forming projecting mold part 11A from the interiors of the fine hollow protrusions 3 to form fine hollow protrusions 3 respectively having hollow interiors as a step following the cooling step. Furthermore, this embodiment includes an opening portion forming step of forming opening portions 3h, which penetrate the interior portions of the fine hollow protrusions 3, at positions offset from the centers of the tip portions of the formed fine hollow protrusions 3 as a step following the release step. Hereinafter, these steps will be specifically described with reference to the drawings.

In this embodiment using the above-described manufacturing apparatus 100, first, a continuous base material sheet 2A containing a thermoplastic resin is paid out from a raw material roll of the base material sheet 2A, and is transported in the Y direction. When the base material sheet 2A has been fed to a predetermined position, transportation of the base material sheet 2A is stopped. In this manner, in this embodiment, the continuous base material sheet 2A is transported intermittently.

Then, in this embodiment, as shown in FIG. 6(a), the protrusion-forming projecting mold part 11A is moved upward at the insertion angle θ1 with respect to the first face 2D (the lower face) of the base material sheet 2A, so that the protrusion-forming projecting mold part 11A is brought into contact with the first face 2D of the continuous base material sheet 2A that is being transported in the Y direction. Here, the insertion angle θ1 is defined as an angle between a bisector passing through the center 11t of the tip portion of a projection 110A of the protrusion-forming projecting mold part 11A that is used in the protrusion forming step and the first face (the lower face) 2D of the base material sheet 2A. In this embodiment, the insertion angle θ1 is 90 degrees and matches the thickness direction (Z direction).

Then, the protrusion-forming projecting mold part 11A is inserted into the base material sheet 2A while softening, by heat, the contact points TP on the base material sheet 2A, and thus the fine hollow protrusions 3 which project from the second face 2U (the upper face) of the base material sheet 2A and which do not penetrate the projecting base material sheet 2A are formed (protrusion forming step). In the protrusion forming step of this embodiment using the manufacturing apparatus 100, as shown in FIG. 4, the opening plate 12U arranged on the second face 2U side (the upper face side) of the continuous base material sheet 2A that has been paid out from the raw material roll and is being transported in the Y direction supports the base material sheet 2A. In this state, the protrusion-forming projecting mold part 11A is moved upward in the thickness direction (Z direction) by the electric actuator (not shown) toward the first face 2D (the lower face) of the base material sheet 2A at a portion thereof corresponding to the opening of the opening plate 12U, and thus the tip portions of the projections 110A of the protrusion-forming projecting mold part 11A are brought into contact with the first face 2D. In this manner, in the protrusion forming step, the second face 2U (the upper face) corresponding to the contact points TP of the base material sheet 2A with which the projections 110A of the protrusion-forming projecting mold part 11A have been brought into contact is not provided with, for example, depressions that are to be fitted to the protrusion-forming projecting mold part 11A for forming protrusions, and are not held down.

In this embodiment, as shown in FIG. 6(a), the ultrasonic vibration device causes the protrusion-forming projecting mold part 11A to vibrate ultrasonically at the contact points TP, and thus the contact points TP are softened by heat generated through friction at the contact points TP. Then, in the protrusion forming step of this embodiment, while the contact points TP are softened, as shown in FIG. 6(b), the protrusion-forming projecting mold part 11A is raised from the first face 2D side (the lower face side) of the base material sheet 2A toward the second face 2U (the upper face), the tip portions of the projections 110A are inserted into the base material sheet 2A, and then the fine hollow protrusions 3 which project from the second face 2U (the upper face) of the base material sheet 2A and which do not penetrate the projecting base material sheet 2A are formed.

In the protrusion forming step of this embodiment, ultrasonic vibrations generated by the ultrasonic vibration device of the protrusion-forming projecting mold part 11A are such that the vibration frequency thereof (hereinafter, referred to as “frequency”) is preferably 10 kHz or greater, and more preferably 15 kHz or greater, is preferably 50 kHz or less, and more preferably 40 kHz or less, and, specifically, is preferably from 10 to 50 kHz, and more preferably from 15 to 40 kHz, in order to form the fine hollow protrusions which project from the base material sheet 2A and which do not penetrate the base material sheet 2A that is projecting.

Furthermore, ultrasonic vibrations generated by the ultrasonic vibration device of the protrusion-forming projecting mold part 11A are such that the amplitude thereof is preferably 1 μm or greater, and more preferably 5 μm or greater, is preferably 60 μm or less, and more preferably 50 μm or less, and, specifically, is preferably from 1 to 60 μm, and more preferably from 5 to 50 μm, in order to form the fine hollow protrusions which project from the base material sheet 2A and which do not penetrate the projecting base material sheet 2A. In the case where an ultrasonic vibration device is used as in this embodiment, in the protrusion forming step, the frequency and the amplitude of ultrasonic vibrations of the protrusion-forming projecting mold part 11A may be adjusted within the above-described range.

In the protrusion forming step of this embodiment, if the insertion speed at which the protrusion-forming projecting mold part 11A is inserted into the base material sheet 2A is too slow, the resin is softened excessively, whereas, if the insertion speed is too fast, softening is insufficient, and the height of the fine hollow protrusions 3 is likely to be insufficient. Thus, in order to efficiently form the fine hollow protrusions 3 which do not penetrate the projecting base material sheet 2A, the insertion speed is preferably 0.1 mm/sec or greater, and more preferably 1 mm/sec or greater, is preferably 1000 mm/sec or less, and more preferably 800 mm/sec or less, and, specifically, is preferably from 0.1 to 1000 mm/sec, and more preferably from 1 to 800 mm/sec.

In the protrusion forming step of this embodiment, the insertion height by which the protrusion-forming projecting mold part 11A is inserted into the base material sheet 2A is preferably 0.01 mm or greater, and more preferably 0.02 mm or greater, is preferably 10 mm or less, and more preferably 5 mm or less, and, specifically, is preferably from 0.01 to 10 mm, and more preferably from 0.02 to 5 mm, in order to efficiently form the fine hollow protrusions 3 which do not penetrate the projecting base material sheet 2A. Here, “insertion height” refers to the distance between the apex of a projection 110A of the protrusion-forming projecting mold part 11A and the second face 2U of the base material sheet 2A in a state where the projection 110A of the protrusion-forming projecting mold part 11A is inserted into the base material sheet 2A. Accordingly, the insertion height in the protrusion forming step refers to the distance from the second face 2U to the apex of a projection 110A as measured in the perpendicular direction in a state where the projection 110A has been inserted to the deepest position and has been arranged in the interior of the fine hollow protrusion 3 projecting from the second face 2U of the base material sheet 2A in the protrusion forming step.

In the protrusion forming step of this embodiment, if the softening time that is the time from when raising of the protrusion-forming projecting mold part 11A in a heated state is stopped until when a cooling step, which is the next step, is performed while keeping the projections 110A of the protrusion-forming projecting mold part 11A inserted in the fine hollow protrusions 3 is too long, the contact points TP on the base material sheet 2A are excessively softened, but, in order to compensate for insufficient softening, the softening time is preferably 0 seconds or greater, and more preferably 0.1 seconds or greater, is preferably 10 seconds or less, and more preferably 5 seconds or less, and, specifically, is preferably from 0 to 10 seconds, and more preferably from 0.1 to 5 seconds.

Next, as shown in FIG. 6(c), the fine hollow protrusions 3 are cooled in a state where the protrusion-forming projecting mold part 11A is inserted in the fine hollow protrusions 3 (cooling step). In the cooling step of this embodiment, the movement in the thickness direction (Z direction) of the protrusion-forming projecting mold part 11A by the electric actuator (not shown) is stopped, and cooling is performed while keeping the projections 110A inserted in the fine hollow protrusions 3 by blowing cold air from the air vent 22 arranged on the second face 2U side (the upper face side) of the base material sheet 2A in a state where the projections 110A of the protrusion-forming projecting mold part 11A are inserted in the fine hollow protrusions 3. Note that, when performing cooling, generation of ultrasonic vibrations by the ultrasonic vibration device of the protrusion-forming projecting mold part 11A may be continued or stopped, but, in order to keep the shape of the fine hollow protrusions 3 constant without it excessively changing, the generation of ultrasonic vibrations is preferably stopped.

The temperature of the cold air to be blown is preferably −50° C. or greater, and more preferably −40° C. or greater, is preferably 26° C. or less, and more preferably 10° C. or less, and, specifically, is preferably from −50 to 26° C., and more preferably from −40 to 10° C., in order to form the fine hollow protrusions 3 which do not penetrate the projecting base material sheet 2A.

The cooling time for cooling by blowing cold air is preferably 0.01 seconds or greater, and more preferably 0.5 seconds or greater, is preferably 60 seconds or less, and more preferably 30 seconds or less, and, specifically, is preferably from 0.01 to 60 seconds, and more preferably from 0.5 to 30 seconds, in order to balance moldability and processing time.

Then, as shown in FIG. 6(d), the protrusion-forming projecting mold part 11A is withdrawn from the interiors of the fine hollow protrusions 3, so that fine hollow protrusions 3 respectively having hollow interiors are formed (release step). In the release step of this embodiment, ultrasonic vibrations generated by the ultrasonic vibration device of the protrusion-forming projecting mold part 11A is stopped, the protrusion-forming projecting mold part 11A is moved downward in the thickness direction (Z direction) by the electric actuator (not shown), the projections 110A are released from the state in which the projections 110A are inserted in the interiors of the fine hollow protrusions 3, and then the fine hollow protrusions 3 respectively having hollow interiors are formed. In this embodiment, nine fine hollow protrusions 3 formed in this manner are arranged in an array on the second face 2U (the upper face) of the base material sheet 2A.

Then, as shown in FIG. 6(e), opening portions 3h, which penetrate the interior portions of the fine hollow protrusions 3, are formed at positions offset from the centers of the tip portions of the formed fine hollow protrusions 3 (opening portion forming step). In the opening portion forming step of this embodiment, an opening-forming projecting mold part 11B that is different from the protrusion-forming projecting mold part 11A is moved downward from the second face 2U side (the upper face side) of the base material sheet 2A at the insertion angle θ2 with respect to the first face (the lower face) 2D of the base material sheet 2A. Here, the insertion angle θ2 is defined as an angle between a bisector passing through the center 11t of the tip portion of a projection 110B of the opening-forming projecting mold part 11B that is used in the opening portion forming step and the first face (the lower face) 2D of the base material sheet 2A. In this embodiment, the insertion angle θ2 is 270 degrees, that is, its difference from the insertion angle θ1 (90 degrees) of the protrusion-forming projecting mold part 11A that is used in the above-described protrusion forming step is 180 degrees.

When the opening-forming projecting mold part 11B is moved downward, the opening-forming projecting mold part 11B is brought into contact with the fine hollow protrusions 3, which do not penetrate the projecting base material sheet 2A, at positions offset from the centers of the tip portions of the fine hollow protrusions 3, the opening-forming projecting mold part 11B is inserted into the fine hollow protrusions 3 while softening, by heat, a contact points TP1 of the fine hollow protrusions 3 which contact with the opening-forming projecting mold part 11B, and thus opening portions 3h which penetrate the interior portions of the fine hollow protrusions 3 are formed. Preferably, in the manufacturing apparatus 100 of this embodiment, as described above, the center 11t1 of a tip portion of the protrusion-forming projecting mold part 11A (the center of the tip portion of the fine hollow protrusion 3 which does not penetrate the base material sheet 2A that is projecting) and the center 11t2 of a tip portion of the opening-forming projecting mold part 11B are offset from each other by an offset amount M1 (see FIG. 6(c)). In the opening portion forming step of this embodiment using the manufacturing apparatus 100, as shown in FIG. 6(e), the opening-forming projecting mold part 11B is moved downward in the thickness direction (Z direction) by the electric actuator (not shown), and is brought into contact with the fine hollow protrusions 3 at positions offset from the centers of the tip portions of the fine hollow protrusions 3, from the second face 2U side of the base material sheet 2A.

In this embodiment, as shown in FIG. 6(e), the ultrasonic vibration device causes the opening-forming projecting mold part 11B to vibrate ultrasonically at the contact points TP1, and thus the contact points TP1 are softened by heat generated through friction at the contact points TP1. Then, in the opening portion forming step of this embodiment, while the contact points TP1 are softened, as shown in FIG. 6(e), the opening-forming projecting mold part 11B is lowered from the second face 2U side (the upper face side) of the base material sheet 2A toward the first face 2D (the lower face), and the tip portions of the projections 110B are inserted into positions offset from the centers of the tip portions of the fine hollow protrusions 3, and then the opening portions 3h that penetrate the interior portions of the fine hollow protrusions 3 projecting from the second face 2U (the upper face) of the base material sheet 2A are formed.

In the opening portion forming step of this embodiment, ultrasonic vibrations generated by the ultrasonic vibration device of the opening-forming projecting mold part 11B are such that the vibration frequency thereof (hereinafter, referred to as “frequency”) is preferably 10 kHz or greater, and more preferably 15 kHz or greater, is preferably 50 kHz or less, and more preferably 40 kHz or less, and, specifically, is preferably from 10 to 50 kHz, and more preferably from 15 to 40 kHz, in order to efficiently form the fine hollow protrusions 3 having the opening portions 3h at positions offset from the centers of the tip portions.

Furthermore, ultrasonic vibrations generated by the ultrasonic vibration device of the opening-forming projecting mold part 11B are such that the amplitude thereof is preferably 1 μm or greater, and more preferably 5 μm or greater, is preferably 60 μm or less, and more preferably 50 μm or less, and, specifically, is preferably from 1 to 60 μm, and more preferably from 5 to 50 μm, in order to efficiently form the fine hollow protrusions 3 having the opening portions 3h at positions offset from the centers of the tip portions. In the case where an ultrasonic vibration device is used as in this embodiment, in the opening portion forming step, the frequency and the amplitude of ultrasonic vibrations of the opening-forming projecting mold part 11B may be adjusted within the above-described range.

In the opening portion forming step of this embodiment, if the insertion speed at which the opening-forming projecting mold part 11B is inserted into the fine hollow protrusions 3 which do not penetrate the projecting base material sheet 2A is too slow, the resin is softened excessively, and the size of the opening portions 3h changes too significantly, whereas, if the insertion speed is too fast, softening is insufficient, and it is difficult to form the opening portions 3h into a desired shape. Thus, in order to efficiently form the fine hollow protrusions 3 having the opening portions 3h at positions offset from the centers of the tip portions, the insertion speed is preferably 0.1 mm/sec or greater, and more preferably 1 mm/sec or greater, is preferably 1000 mm/sec or less, and more preferably 800 mm/sec or less, and, specifically, is preferably from 0.1 to 1000 mm/sec, and more preferably from 1 to 800 mm/sec.

In the opening portion forming step of this embodiment, the frequency and the amplitude of ultrasonic vibrations of the opening-forming projecting mold part 11B generated by the ultrasonic vibration device are the same as the frequency and the amplitude of ultrasonic vibrations of the protrusion-forming projecting mold part 11A that is used in the protrusion forming step.

Meanwhile, in the opening portion forming step of this embodiment, the insertion speed at which the opening-forming projecting mold part 11B is inserted into the fine hollow protrusions 3 which do not penetrate the projecting base material sheet 2A is faster than the insertion speed at which the protrusion-forming projecting mold part 11A is inserted into the base material sheet 2A in the protrusion forming step. In this embodiment, the heating means (not shown) of each of the projecting mold parts 11A and 11B is an ultrasonic vibration device, wherein the frequency and the amplitude of ultrasonic vibrations of the protrusion-forming projecting mold part 11A included in the protrusion forming section 10 are the same as the frequency and the amplitude of ultrasonic vibrations of the opening-forming projecting mold part 11B included in the opening portion forming section 9, that is, (Condition b) and (Condition c) described above are not satisfied. However, in this embodiment, the insertion speed of the protrusion-forming projecting mold part 11A into the base material sheet 2A in the protrusion forming step is slower than the insertion speed of the opening-forming projecting mold part 11B into the fine hollow protrusions 3 in the opening portion forming step, that is, (Condition a) described above is satisfied. Accordingly, the amount of processing heat that is applied from the protrusion-forming projecting mold part 11A to the base material sheet 2A in the protrusion forming step is larger than the amount of processing heat that is applied from the opening-forming projecting mold part 11B to the fine hollow protrusions 3 in the opening portion forming step. Accordingly, it is possible to precisely form fine hollow protrusions 3 having the opening portions 3h at positions offset from the centers of the tip portions.

Then, as shown in FIG. 6(f), the opening-forming projecting mold part 11B is moved upward in the thickness direction (Z direction) by the electric actuator (not shown), the opening-forming projecting mold part 11B inserted into the fine hollow protrusions 3 is withdrawn, and then a precursor 1A of the microneedle arrays 1M is formed. In the thus formed precursor 1A of a continuous fine hollow protruding tool that is to be formed into the microneedle arrays 1M, nine fine hollow protrusions 3 having the opening portions 3h at positions offset from the centers of the tip portions are arranged in an array.

The thus formed precursor 1A of the microneedle arrays 1M is then transported downstream in the transporting direction (Y direction). Then, the precursor 1A is cut in a predetermined range in a cutting step, and thus a microneedle array 1M can be manufactured as the fine hollow protruding tool 1 of this embodiment including a sheet-like basal member 2 and a plurality of fine hollow protrusions 3 as shown in FIG. 1. Fine hollow protruding tools 1 can be continuously and efficiently manufactured on the second face 2U side (the upper face side) of the base material sheet 2A, by repeating the above-described steps.

The microneedle array 1M manufactured as described above may be further shaped into a predetermined shape in subsequent steps, or the shape of the base material sheet 2A may be adjusted in advance into a desired shape before the step of inserting the protrusion-forming projecting mold part 11A.

As described above, the manufacturing method of this embodiment for manufacturing the microneedle array 1M includes the protrusion forming step of forming the fine hollow protrusions 3 which do not penetrate the projecting base material sheet 2A using the protrusion-forming projecting mold part 11A including the heating means, the cooling step of performing cooling in a state where the protrusion-forming projecting mold part 11A is inserted in the fine hollow protrusions 3, and the release step of withdrawing the protrusion-forming projecting mold part 11A to form the fine hollow protrusions 3 respectively having hollow interiors, and further includes the opening portion forming step of forming opening portions 3h, which penetrate the interior portions of the fine hollow protrusions 3, at positions offset from the centers of the tip portions of the formed fine hollow protrusions 3, as a step following the release step. The manufacturing method of this embodiment includes the protrusion forming step, the cooling step, the release step, and the opening portion forming step in this order, and thus it is possible to manufacture the fine hollow protruding tool 1 with a precise shape having the opening portions 3h at positions offset from the centers of the tip portions. Furthermore, the thus formed microneedle array 1M has the opening portions 3h at positions offset from the centers of the tip portions of the fine hollow protrusions 3, and thus, when piercing the skin, the opening portions 3h are unlikely to be crushed, and it is possible to stably deliver agents into the skin. According to the manufacturing method of this embodiment, it is possible to form the fine hollow protrusions 3 with simple steps using the projecting mold parts 11A and 11B including the heating means, and thus, it is possible to efficiently manufacture the microneedle array 1M that can stably deliver agents into the skin, which reduces the cost.

Furthermore, in the opening portion forming step of this embodiment, the opening portions 3h are formed using the opening-forming projecting mold part 11B having a heating means (not shown). Accordingly, it is possible to form the opening portions 3h which penetrate the interior portions of the fine hollow protrusions 3, without damaging to the extent possible the moldability of the fine hollow protrusions 3 formed in the preceding protrusion forming step, and it is possible to manufacture the fine hollow protruding tool 1 with a more precise shape having the opening portions 3h at positions offset from the centers of the tip portions.

Furthermore, in this embodiment, an ultrasonic vibration device is used as the heating means (not shown) of each of the projecting mold parts 11A and 11B, and thus the cold air blowing device 21 does not necessarily have to be included, and it is also possible to perform cooling merely by turning off vibrations of the ultrasonic vibration device. According to this aspect, if an ultrasonic vibration is used as the heating means, it is possible to simplify the apparatus, and to manufacture the microneedle array 1M having the opening portions 3h at high speed.

Furthermore, in this embodiment, the insertion angle θ1 of the protrusion-forming projecting mold part 11A that is used in the protrusion forming step with respect to the first face 2D of the base material sheet 2A is different from the insertion angle θ2 of the opening-forming projecting mold part 11B that is used in the opening portion forming step with respect to the first face 2D of the base material sheet 2A. If the insertion angles are different in this manner, it is easy to form the opening portions 3h at positions offset from the centers of the tip portions of the fine hollow protrusions 3, and it is possible to manufacture the fine hollow protruding tool 1 with a more precise shape having the opening portions 3h at positions offset from the centers of the tip portions.

Furthermore, in this embodiment, the protrusion-forming projecting mold part 11A that is used in the protrusion forming step is brought into contact with the first face 2D of the base material sheet 2A, and the opening-forming projecting mold part 11B that is used in the opening portion forming step is brought into contact with the second face 2U of the base material sheet 2A. Accordingly, it is easy to form the opening portions 3h at positions offset from the centers of the tip portions of the fine hollow protrusions 3, and it is possible to manufacture the fine hollow protruding tool 1 with a more precise shape having the opening portions 3h at positions offset from the centers of the tip portions.

Furthermore, in this embodiment, the protrusion-forming projecting mold part 11A is different from the opening-forming projecting mold part 11B. Accordingly, the degree of freedom in the shape of the opening portions 3h and the degree of freedom in the shape of the fine hollow protruding tool 1 are improved, and the processability is improved.

Furthermore, as described above, in this embodiment, the ultrasonic vibration device causes the projecting mold parts 11A and 11B to vibrate only at the contact points TP of the base material sheet 2A with which the protrusion-forming projecting mold part 11A has been brought into contact as shown in FIG. 6(a) and only at the contact points TP1 of the fine hollow protrusions 3 with which the opening-forming projecting mold part 11B, which is different from the protrusion-forming projecting mold part 11A, has been brought into contact as shown in FIG. 6(e) so that the contact points TP and TP1 soften, and thus it is possible to efficiently and continuously manufacture the microneedle arrays 1M having the opening portions 3h at low energy.

Furthermore, as described above, the manufacturing apparatus 100 of this embodiment is such that, in the protrusion forming section 10, the operation of the protrusion-forming projecting mold part 11A, the heating conditions of the heating means (not shown) of the protrusion-forming projecting mold part 11A, the softening time of the contact points TP of the base material sheet 2A, and the insertion speed of the protrusion-forming projecting mold part 11A into the base material sheet 2A can be adjusted by a control means (not shown). Furthermore, the cooling temperature and the cooling time of the cold air blowing device 21 of the cooling section 20 are controlled by a control means (not shown). Furthermore, in the opening portion forming section 9, the operation of the opening-forming projecting mold part 11B, the heating conditions of the heating means (not shown) of the opening-forming projecting mold part 11B, the softening time of the contact points TP1 of the fine hollow protrusions 3, and the insertion speed of the opening-forming projecting mold part 11B into the fine hollow protrusions 3 can be adjusted. Accordingly, it is possible to freely control the shape of the microneedle array 1M having the opening portions 3h, using the control means (not shown).

Furthermore, according to the fine hollow protruding tool 1 having the rising portions 4 at the peripheral edges of the opening portions 3h formed by the above-described manufacturing method, it is possible stably deliver agents into the skin through the opening portions that are unlikely to be crushed when piercing the skin.

Above, the present invention has been described based on a preferable embodiment, but the present invention is not limited to the foregoing embodiment, and may be changed as appropriate.

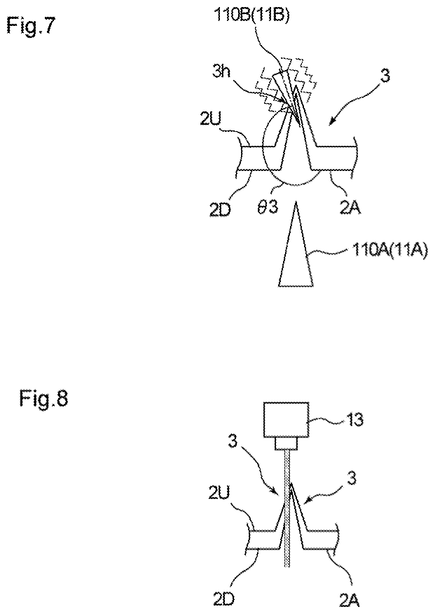

For example, in the method for manufacturing the microneedle array 1M according to the foregoing embodiment, the insertion angle θ1 of the protrusion-forming projecting mold part 11A with respect to the base material sheet 2A is different from the insertion angle θ2 of the opening-forming projecting mold part 11B with respect to the base material sheet 2A. Specifically, a difference between the insertion angle θ1 of the protrusion-forming projecting mold part 11A with respect to the first face (the lower face) 2D of the base material sheet 2A and the insertion angle θ2 of the opening-forming projecting mold part 11B with respect to the first face (the lower face) 2D of the base material sheet 2A is 180 degrees. However, the difference may not be 180 degrees. For example, the difference between the insertion angle θ1 (see FIG. 6(a)) of the protrusion-forming projecting mold part 11A with respect to the first face (the lower face) 2D of the base material sheet 2A and an insertion angle θ3 of the opening-forming projecting mold part 11B with respect to the base material sheet 2A may be greater than 90 degrees and less than 180 degrees as shown in FIG. 7.

Also in the case where the difference between the insertion angle θ1 of the protrusion-forming projecting mold part 11A with respect to the base material sheet 2A in the protrusion forming step and the insertion angle θ3 of the opening-forming projecting mold part 11B with respect to the base material sheet 2A in the opening portion forming step is greater than 90 degrees and less than 180 degrees in this manner, it is possible to form the opening portions 3h at positions offset from the centers of the tip portions of the fine hollow protrusions 3, and it is possible to efficiently manufacture the microneedle array 1M with a precise shape having the opening portions 3h at positions offset from the centers of the tip portions of the fine hollow protrusions 3. Furthermore, the degree of freedom in the shape of the opening portions 3h is improved, and the processability can be improved.

Furthermore, although the method for manufacturing the microneedle array 1M according to the foregoing embodiment was described using the opening-forming projecting mold part 11B having the conical projections 110B, the shape of the projections 110B of the opening-forming projecting mold part 11B is not limited to a conical shape, and it may be a pyramidal shape, a cylindrical shape, a prismatic shape, or the like. Furthermore, in the method for manufacturing the microneedle array 1M according to the foregoing embodiment, the projections 110B of the opening-forming projecting mold part 11B that is used in the opening portion forming step are in a bilaterally symmetrical conical shape when viewed in a vertical cross-section, but they may be in a bilaterally asymmetrical shape when viewed in a vertical cross-section.

Also in the case where the opening-forming projecting mold part 11B has the projections 110B that are in a pyramidal shape, a cylindrical shape, a prismatic shape, or a bilaterally asymmetrical shape when viewed in a vertical cross-section, it is possible to form opening portions 3h which penetrate the interior portions of the fine hollow protrusions 3 which do not penetrate the projecting base material sheet 2A, by using the ultrasonic vibration device to cause the opening-forming projecting mold part 11B to vibrate ultrasonically, bringing the projecting mold part 11B into contact with the fine hollow protrusions 3, which do not penetrate the projecting base material sheet 2A, at positions offset from the centers of the tip portions of the fine hollow protrusions 3, and inserting the projecting mold part 11B into the fine hollow protrusions 3 while softening, by heat, contact points TP1.

Furthermore, in the opening portion forming step of the method for manufacturing the microneedle array 1M according to the foregoing embodiment, the opening portions 3h are formed using the opening-forming projecting mold part 11B including the heating means, but it is also possible to form opening portions 3h which penetrate the interior portions of the fine hollow protrusions 3 which do not penetrate the projecting base material sheet 2A, at positions offset from the centers of the tip portions of the fine hollow protrusions 3 which do not penetrate the projecting base material sheet 2A, from the second face 2U side (the upper face side) toward the first face 2D (the lower face), using a non-contact thermal processing means. For example, it is possible to form opening portions 3h using a laser irradiation device 13 as shown in FIG. 8. The non-contact thermal processing means may be, for example, a hot air jetting device for jetting hot air or the like instead of the laser irradiation device 13. Also in the case where a non-contact thermal processing means is used, preferably, it is possible to form opening portions 3h in the base material sheet 2A in the opening portion forming step.

If a non-contact thermal processing means is used, for example, the level of precision does not decrease due to wearing down or the like even when it is used over a long period of time, and thus it is possible to efficiently manufacture the microneedle array 1M with a precise shape having the opening portions 3h. Furthermore, if a non-contact thermal processing means is used, the degree of freedom in the shape of the opening portions 3h can be increased.

Furthermore, in the opening portion forming step of the method for manufacturing the microneedle array 1M according to the foregoing embodiment, one opening portion 3h is formed by the opening-forming projecting mold part 11B at a position offset from the center of the tip portion of the fine hollow protrusion 3 which do not penetrate the projecting base material sheet 2A, but, for example, it is also possible to form a plurality of opening portions 3h at positions offset from the center of the tip portion of the fine hollow protrusion 3 which do not penetrate the projecting base material sheet 2A.

If a plurality of opening portions 3h are formed at positions offset from the center of the tip portion of the fine hollow protrusion 3 which do not penetrate the projecting base material sheet 2A in this manner, the liquid pressure inside the fine hollow protrusion 3 when delivering agents can be lowered, and thus the risk that the opening portions will be blocked can be reduced, and the liquid injection efficiency can be improved.

Note that an opening portion 3h is arranged at a position offset from the tip portion of the fine hollow protrusion 3, in a direction toward the base portion, preferably by 2% or greater than the height H1 of the fine hollow protrusion 3, more preferably by 5% or greater than the height H1, and even more preferably by 10% or greater than the height H1. Furthermore, the opening portion 3h is arranged at a position offset from the base portion of the fine hollow protrusion 3, in a direction toward the tip portion, preferably by 2% or greater than the height H1 of the fine hollow protrusion 3, more preferably by 5% or greater than the height H1, and even more preferably by 10% or greater than the height H1.

Furthermore, in the method for manufacturing the microneedle array 1M according to the foregoing embodiment, the frequency and the amplitude of ultrasonic vibrations of the opening-forming projecting mold part 11B are the same as the frequency and the amplitude of ultrasonic vibrations of the protrusion-forming projecting mold part 11A, that is, (Condition b) and (Condition c) are not satisfied. However, the insertion speed into the base material sheet 2A is such that the insertion speed of the protrusion-forming projecting mold part 11A is slower than the insertion speed of the opening-forming projecting mold part 11B, and thus (Condition a) is satisfied. As a result, the amount of processing heat that is applied from the protrusion-forming projecting mold part 11A to the base material sheet 2A in the protrusion forming step is larger than the amount of processing heat that is applied from the opening-forming projecting mold part 11B to the base material sheet 2A in the opening portion forming step.

That is to say, the method for manufacturing the microneedle array 1M according to the foregoing embodiment is such that, regarding a difference between the processing conditions by the opening-forming projecting mold part 11B and the processing conditions by the protrusion-forming projecting mold part 11A, the conditions of the heating means included in the opening-forming projecting mold part 11B in the opening portion forming step are the same as the conditions of the heating means included in the protrusion-forming projecting mold part 11A in the protrusion forming step, but the speed at which the protrusion-forming projecting mold part 11A is inserted into the base material sheet 2A in the protrusion forming step is slower than the speed at which the opening-forming projecting mold part 11B is inserted into the base material sheet 2A in the opening portion forming step.

However, it is also possible that the method for manufacturing the microneedle array 1M is a manufacturing method in which the speed at which the opening-forming projecting mold part 11B is inserted into the base material sheet 2A in the opening portion forming step is the same as the speed at which the protrusion-forming projecting mold part 11A is inserted into the base material sheet 2A in the protrusion forming step, but the amount of processing heat that is applied to the base material sheet 2A under the conditions of the heating means included in the protrusion-forming projecting mold part 11A in the protrusion forming step is larger than the amount of processing heat that is applied to the base material sheet 2A under the conditions of the heating means included in the opening-forming projecting mold part 11B in the opening portion forming step. Specifically, it is also possible that, although (Condition a) is not satisfied, the frequency or the amplitude of ultrasonic vibrations of the protrusion-forming projecting mold part 11A is larger than the frequency or the amplitude of ultrasonic vibrations of the opening-forming projecting mold part 11B, that is, (Condition b) or (Condition c) is satisfied, as a result of which, the amount of processing heat that is applied from the protrusion-forming projecting mold part 11A to the base material sheet 2A is larger than the amount of processing heat that is applied from the opening-forming projecting mold part 11B to the base material sheet 2A.

Furthermore, although the method for manufacturing the microneedle array 1M according to the foregoing embodiment was described using an ultrasonic vibration device as the heating means of the projecting mold parts 11A and 11B, the heating means of the projecting mold parts 11A and 11B may also be a heater device.

In the manufacturing method according to the foregoing embodiment in which the heating means of the projecting mold part 11 is a heater device, when the heater temperature of the protrusion-forming projecting mold part 11A is the same as the heater temperature of the opening-forming projecting mold part 11B, (Condition d) is not satisfied, but, if the insertion speed of the protrusion-forming projecting mold part 11A in the protrusion forming step is slower than the insertion speed of the opening-forming projecting mold part 11B in the opening portion forming step, (Condition a) is satisfied. As a result, the amount of processing heat that is applied from the protrusion-forming projecting mold part 11A to the base material sheet 2A in the protrusion forming step is larger than the amount of processing heat that is applied from the opening-forming projecting mold part 11B to the base material sheet 2A in the opening portion forming step. Furthermore, it is also possible that, although (Condition a) is not satisfied, the heater temperature of the protrusion-forming projecting mold part 11A is higher than the heater temperature of the opening-forming projecting mold part 11B, that is, (Condition d) is satisfied, as a result of which, the amount of processing heat that is applied from the protrusion-forming projecting mold part 11A to the base material sheet 2A in the protrusion forming step is larger than the amount of processing heat that is applied from the opening-forming projecting mold part 11B to the base material sheet 2A in the opening portion forming step. Furthermore, all of (Condition a), (Condition b), (Condition c), and (Condition d) described above may be satisfied.

The heating temperature of the base material sheet 2A due to each of the projecting mold parts 11A and 11B is preferably a temperature that is the same as or greater than the glass transition temperature and that is below the melting temperature of the base material sheet 2A, and more preferably a temperature that is the same as or greater than the softening temperature and that is below the melting temperature. Specifically, the heating temperature is preferably 30° C. or greater, and more preferably 40° C. or greater, is preferably 300° C. or less, and more preferably 250° C. or less, and, specifically, is preferably from 30 to 300° C., and more preferably from 40 to 250° C. If the base material sheet 2A is heated using an ultrasonic vibration device, the aforementioned heating temperature is employed as the temperature range of a portion of the base material sheet 2A that comes into contact with the projections 110. Meanwhile, if a heater device is used, the heating temperature of the projecting mold part 11 may be adjusted within the aforementioned range.

It should be noted that the glass transition temperature (Tg) is measured according to the following measurement method, and the softening temperature is measured according to JIS K-7196 “Testing method for softening temperature of thermoplastic film and sheeting by thermomechanical analysis”.

Note that the “glass transition temperature (Tg) of the base material sheet 2A” refers to the glass transition temperature (Tg) of the resin constituting the base material sheet 2A. In cases where there are a plurality of types of constituent resins and the plurality of glass transition temperatures (Tg) are different from each other, the heating temperature of the base material sheet 2A by the heating means is preferably at least equal to or higher than the lowest glass transition temperature (Tg) among the plurality of glass transition temperatures (Tg), and more preferably equal to or higher than the highest glass transition temperature (Tg) among the plurality of glass transition temperatures (Tg).

The same applies to the “softening temperature of the base material sheet 2A”, as with the glass transition temperature (Tg). In cases where there are a plurality of types of constituent resins in the base material sheet 2A and the plurality of softening temperatures are different from each other, the heating temperature of the base material sheet 2A by the heating means is preferably at least equal to or higher than the lowest softening temperature among the plurality of softening temperatures, and more preferably equal to or higher than the highest softening temperature among the plurality of softening temperatures.

In cases where the base material sheet 2A includes two or more types of resins having different melting points, the heating temperature of the base material sheet 2A by the heating means is preferably below the lowest melting point among the plurality of melting points.

### Method for Measuring Glass Transition Temperature (Tg)

The glass transition temperature is determined by measuring the heat quantity by using a DSC measurement device. More specifically, the measurement device used is a differential scanning calorimeter (Diamond DSC) from Perkin Elmer. A 10-mg test piece is sampled from the base sheet. As for the measurement conditions, the temperature is kept constant at 20° C. for 5 minutes, and then the temperature is raised from 20° C. to 320° C. at a rate of 5° C./minute, to obtain a DSC curve wherein the horizontal axis indicates temperature and the vertical axis indicates heat quantity. The glass transition temperature Tg is determined from the DSC curve.

Furthermore, the method for manufacturing the microneedle array 1M of this embodiment was described using a method for manufacturing the microneedle array 1M in which nine truncated conical fine hollow protrusions 3 are arranged in an array on the upper face of the sheet-like basal member 2, but it is also possible to apply this method as a method for manufacturing the microneedle array 1M having one fine hollow protrusion 3.

Furthermore, the method for manufacturing the microneedle array 1M according to the foregoing embodiment was described using a configuration in which the projecting mold part 11 can be moved vertically in the thickness direction (Z direction) by an electric actuator (not shown), but it is also possible to employ a configuration in which movement of the projecting mold part 11 vertically in the thickness direction (Z direction) is realized by using a projecting mold part 11 of a box-motion type that follow an endless track.

Furthermore, the method for manufacturing the microneedle array 1M according to the foregoing embodiment was described using a method for manufacturing the microneedle array 1M including the fine hollow protrusions 3 respectively having the rising portions 4 rising in the shape of convex curves toward the interiors of the fine hollow protrusions 3 at the peripheral edges of the opening portions 3h, but it is also possible to employ a method for manufacturing the fine hollow protruding tool of the present invention in which a fine hollow protruding tool 1 having no rising portion 4 at the peripheral edges of the opening portions 3h is manufactured.

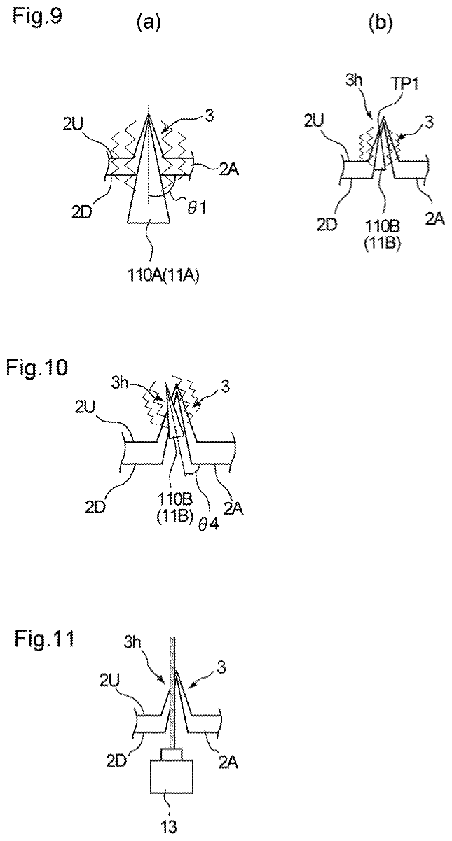

According to the method for manufacturing the microneedle array 1M as the fine hollow protruding tool 1 having no rising portion 4 at the peripheral edges of the opening portions 3h, after the protrusion forming step shown in FIG. 9(a), in the opening portion forming step, as shown in FIG. 9(b), an opening-forming projecting mold part 11B that is different from the protrusion-forming projecting mold part 11A is moved upward in the thickness direction (Z direction) from the first face 2D side (the lower face side) of the base material sheet 2A toward the second face 2U (the upper face) in a state where the ultrasonic vibration device causes the opening-forming projecting mold part 11B to vibrate ultrasonically. Then, the opening-forming projecting mold part 11B is inserted from the interior of each fine hollow protrusion 3, which does not penetrate the projecting base material sheet 2A, formed in the protrusion forming step and is brought into contact with the interior of the fine hollow protrusion 3, which does not penetrate the projecting base material sheet 2A, at a position offset from the center of the tip portion of the fine hollow protrusion 3, and the contact point TP1 is softened by heat generated through friction at the contact point TP1. While the contact point TP1 is softened, the opening-forming projecting mold part 11B is raised from the first face 2D side (the lower face side) of the base material sheet 2A toward the second face 2U (the upper face), and the tip portion of the projection 110B is inserted into the position offset from the center of the tip portion of the fine hollow protrusion 3, and thus the opening portion 3h which penetrates the interior portion of the fine hollow protrusion 3 toward the outside is formed.

In this manner, in the opening portion forming step, the opening-forming projecting mold part 11B is moved at the same insertion angle as the protrusion-forming projecting mold part 11A from the first face (the lower face) 2D side of the base material sheet 2A, the moving direction of the opening-forming projecting mold part 11B is the same direction as the protrusion-forming projecting mold part 11A, the tip portions of the projections 110B is inserted into positions offset from the centers of the tip portions of the fine hollow protrusions 3 from the interiors of the fine hollow protrusions 3 which do not penetrate the projecting base material sheet 2A, and then opening portions 3h are formed.

In the case where opening portions 3h are formed by the opening-forming projecting mold part 11B, in the opening portion forming step, as shown in FIGS. 9(a) and 9(b), the insertion angle θ1 of the protrusion-forming projecting mold part 11A with respect to the base material sheet 2A may be the same as the insertion angle of the opening-forming projecting mold part 11B with respect to the base material sheet 2A. However, as shown in FIGS. 9(a) and 10, it is also possible that the insertion angle θ1 of the protrusion-forming projecting mold part 11A with respect to the base material sheet 2A is different from an insertion angle θ4 of the opening-forming projecting mold part 11B with respect to the base material sheet 2A. For example, as shown in FIG. 10, it is also possible that the insertion angle θ4 of the opening-forming projecting mold part 11B with respect to the base material sheet 2A is less than 90 degrees.

Also in the case where the insertion angle θ1 of the protrusion-forming projecting mold part 11A with respect to the base material sheet 2A is different from the insertion angle θ4 of the opening-forming projecting mold part 11B with respect to the base material sheet 2A when forming opening portions 3h from the interiors of the fine hollow protrusions 3, which do not penetrate the projecting base material sheet 2A, with the opening-forming projecting mold part 11B in this manner, it is possible to form the opening portions 3h at positions offset from the centers of the tip portions of the fine hollow protrusions 3, and thus it is possible to efficiently manufacture the microneedle array 1M with a precise shape having the opening portions 3h at positions offset from the centers of the tip portions of the fine hollow protrusions 3.

Furthermore, if the insertion angle θ1 of the protrusion-forming projecting mold part 11A with respect to the base material sheet 2A is different from the insertion angle θ4 of the opening-forming projecting mold part 11B with respect to the base material sheet 2A, the degree of freedom in the shape of the opening portions 3h is improved, and the processability can be improved.

Furthermore, if the opening portions 3h are formed in the fine hollow protrusions 3 from the interiors of the fine hollow protrusions 3, which do not penetrate the projecting base material sheet 2A, in the opening portion forming step, the protrusion-forming projecting mold part 11A and the opening-forming projecting mold part 11B may be different projecting mold parts, or may be the same projecting mold part.

Furthermore, if the opening portions 3h are formed from the interiors of the fine hollow protrusions 3, which do not penetrate the projecting base material sheet 2A, at positions offset from the centers of the tip portions of the fine hollow protrusions 3 in the opening portion forming step as described above, it is possible to form the opening portions 3h using the opening-forming projecting mold part 11B including the heating means, but it is also possible to form opening portions 3h which penetrate the interior portions of the fine hollow protrusions 3, which do not penetrate the projecting base material sheet 2A, at positions offset from the centers of the tip portions of the fine hollow protrusions 3, which do not penetrate the projecting base material sheet 2A, using a non-contact thermal processing means instead of the opening-forming projecting mold part 11B including the heating means. For example, it is possible to form opening portions 3h using a laser irradiation device 13 as shown in FIG. 11. The non-contact thermal processing means may be, for example, a hot air jetting device for jetting hot air or the like instead of the laser irradiation device 13. Also in the case where a non-contact thermal processing means is used, preferably, it is possible to form opening portions 3h through the fine hollow protrusions 3, which do not penetrate the projecting base material sheet 2A, in the opening portion forming step.

If a non-contact thermal processing means is used, for example, the level of precision does not decrease due to wearing down or the like even when it is used over a long period of time, and thus it is possible to efficiently manufacture the microneedle array 1M with a precise shape having the opening portions 3h. Furthermore, if a non-contact thermal processing means is used, the degree of freedom in the shape of the opening portions 3h can be increased.

Furthermore, also in the case of the fine hollow protruding tool 1 having no rising portion 4 at the peripheral edges of the opening portions 3h formed by the above-described manufacturing method, it is possible stably deliver agents into the skin through the opening portions that are unlikely to be crushed when piercing the skin.

Furthermore, in the method for manufacturing the microneedle array 1M according to the foregoing embodiment, the protrusion-forming projecting mold part 11A is inserted from the first face 2D of the base material sheet 2A toward the second face 2U in the protrusion forming step, but the positional relationship of the protrusion-forming projecting mold part 11A or the support 12 (the opening plates 12U and 12D) relative to the base material sheet 2A and the insertion direction in the protrusion forming step are not limited thereto, and it is also possible that the insertion direction of the protrusion-forming projecting mold part 11A is a direction from the second face 2U of the base material sheet 2A toward the first face 2D.

For the embodiment described above, the present invention further discloses the following methods for manufacturing a fine hollow protruding tool having an opening portion.

<1>

A method for manufacturing a fine hollow protruding tool, comprising:

a protrusion forming step of contacting a protrusion-forming projecting mold part including a heating means with a first face of a base material sheet containing a thermoplastic resin, and inserting the protrusion-forming projecting mold part into the base material sheet toward a second face of the base material sheet while softening, by heat, the contact portion to form a fine hollow protrusion which projects from the second face of the base material sheet and which does not penetrate the projecting base material sheet;

a cooling step of cooling the fine hollow protrusion in a state where the protrusion-forming projecting mold part is inserted in the fine hollow protrusion;

a release step, as a step following the cooling step, of withdrawing the protrusion-forming projecting mold part from the interior of the fine hollow protrusion to form the fine hollow protrusion having a hollow interior; and

an opening portion forming step of forming an opening portion, which penetrates an interior portion of the fine hollow protrusion, at a position offset from a center of a tip portion of the fine hollow protrusion.

<2>

The method for manufacturing a fine hollow protruding tool as set forth in clause <1>,

wherein the opening portion forming step is performed using an opening-forming projecting mold part including a heating means, and

in the opening portion forming step, the opening portion which penetrates the interior portion of the fine hollow protrusion is formed by contacting the opening-forming projecting mold part with the fine hollow protrusion at a position offset from the center of the tip portion, and inserting the opening-forming projecting mold part into the fine hollow protrusion while softening, by heat, the contact portion.

<3>

The method for manufacturing a fine hollow protruding tool as set forth in clause <2>, wherein a condition of an amount of processing heat in the protrusion forming step is different from a condition of an amount of processing heat in the opening portion forming step.

<4>

The method for manufacturing a fine hollow protruding tool s set forth in clause <3>, wherein a method for making the amounts of processing heat different from each other is satisfying at least one of (Condition a) to (Condition d) below:

(Condition a) an insertion speed of the protrusion-forming projecting mold part into the base material sheet and an insertion speed of the opening-forming projecting mold part into the fine hollow protrusion are such that the insertion speed in the protrusion forming step is slower than the insertion speed in the opening portion forming step;

(Condition b) in a case where the heating means of each projecting mold part is an ultrasonic vibration device, a frequency of ultrasonic waves in the protrusion-forming projecting mold part is higher than a frequency of ultrasonic waves in the opening-forming projecting mold part;

(Condition c) in a case where the heating means of each projecting mold part is an ultrasonic vibration device, an amplitude of ultrasonic waves in the protrusion-forming projecting mold part is larger than an amplitude of ultrasonic waves in the opening-forming projecting mold part; and

(Condition d) in a case where the heating means of each projecting mold part is a heater device, a heater temperature of the protrusion-forming projecting mold part is higher than a heater temperature of the opening-forming projecting mold part.

<5>

The method for manufacturing a fine hollow protruding tool as set forth in any one of clauses <1> to <4>, wherein the heating means is an ultrasonic vibration device.

<6>

The method for manufacturing a fine hollow protruding tool as set forth in any one of clauses <2> to <5>, wherein an insertion angle of the protrusion-forming projecting mold part with respect to the base material sheet in the protrusion forming step is different from an insertion angle of the opening-forming projecting mold part with respect to the base material sheet in the opening portion forming step.

<7>

The method for manufacturing a fine hollow protruding tool as set forth in any one of clauses <2> to <6>, wherein, in the protrusion forming step, the protrusion-forming projecting mold part is brought into contact with the first face side of the base material sheet, and, in the opening portion forming step, the opening-forming projecting mold part is brought into contact with the second face side of the base material sheet.

<8>

The method for manufacturing a fine hollow protruding tool as set forth in any one of clauses <2> to <7>, wherein the protrusion-forming projecting mold part is different from the opening-forming projecting mold part.

<9>

The method for manufacturing a fine hollow protruding tool as set forth in clause <1>, wherein, in the opening portion forming step, the opening portion is formed at a position offset from the center of the tip portion of the fine hollow protrusion, using a non-contact thermal processing means.

<10>

The method for manufacturing a fine hollow protruding tool as set forth in any one of clauses <1> to <9>, wherein, in the opening portion forming step, a plurality of the opening portions are formed at positions offset from the center of the tip portion of the formed fine hollow protrusion.

<11>

The method for manufacturing a fine hollow protruding tool as set forth in any one of clauses <2> to <10>, wherein no heating means is provided other than the heating means of the protrusion-forming projecting mold part and the opening-forming projecting mold part.

<12>

The method for manufacturing a fine hollow protruding tool as set forth in any one of clauses <1> to <11>, wherein a projection of the protrusion-forming projecting mold part has an outer shape that is sharper than an outer shape of the fine hollow protrusion.

<13>

The method for manufacturing a fine hollow protruding tool as set forth in any one of clauses <1> to <12>, wherein a projection of the protrusion-forming projecting mold part has a height that is higher than a height of the fine hollow protruding tool that is to be manufactured, and that is preferably from 0.01 to 30 mm, and more preferably from 0.02 to 20 mm.

<14>

The method for manufacturing a fine hollow protruding tool as set forth in any one of clauses <1> to <13>, wherein a projection of the protrusion-forming projecting mold part has a tip diameter that is preferably from 0.001 to 1 mm, and more preferably from 0.005 to 0.5 mm.

<15>

The method for manufacturing a fine hollow protruding tool as set forth in any one of clauses <1> to <14>, wherein a projection of the protrusion-forming projecting mold part has a base diameter that is preferably from 0.1 to 5 mm, and more preferably from 0.2 to 3 mm.

<16>

The method for manufacturing a fine hollow protruding tool as set forth in any one of clauses <1> to <15>, wherein a projection of the protrusion-forming projecting mold part has a tip angle that is preferably from 1 to 60 degrees, and more preferably from 5 to 45 degrees.

<17>

The method for manufacturing a fine hollow protruding tool as set forth in any one of clauses <1> to <16>, wherein, in the protrusion forming step, a support that supports the base material sheet is provided on the second face side.

<18>

The method for manufacturing a fine hollow protruding tool as set forth in clause <17>, wherein an opening plate having a plurality of openings into which the projection of the protrusion-forming projecting mold part can be inserted is used as the support.

<19>

The method for manufacturing a fine hollow protruding tool as set forth in any one of clauses <17> or <18>, wherein, in the opening portion forming step, a support that supports the base material sheet is provided on the first face side of the base material sheet.

<20>

The method for manufacturing a fine hollow protruding tool as set forth in clause <19>, wherein the support provided on the first face side is an opening plate.

<21>

The method for manufacturing a fine hollow protruding tool as set forth in any one of clauses <1> to <20>, wherein, in the protrusion forming step, an insertion speed at which the protrusion-forming projecting mold part is inserted into the base material sheet is preferably from 0.1 to 1000 mm/sec, and more preferably from 1 to 800 mm/sec.

<22>

The method for manufacturing a fine hollow protruding tool as set forth in any one of clauses <2> to <21>, wherein, in the protrusion forming step, an insertion height by which the protrusion-forming projecting mold part is inserted into the base material sheet is preferably from 0.01 to 10 mm, and more preferably from 0.02 to 5 mm.

<23>

The method for manufacturing a fine hollow protruding tool as set forth in any one of clauses <1> to <22>, wherein an insertion speed at which the opening-forming projecting mold part is inserted into the fine hollow protrusion which does not penetrate the projecting base material sheet is from 0.1 to 1000 mm/sec, and more preferably from 1 to 800 mm/sec.

<24>

The method for manufacturing a fine hollow protruding tool as set forth in any one of clauses <1> to <23>, wherein a heating temperature of the base material sheet due to the protrusion-forming projecting mold part is the same as or greater than a glass transition temperature and is below a melting temperature of the base material sheet, and is preferably the same as or greater than the softening temperature and is below the melting temperature.

<25>

The method for manufacturing a fine hollow protruding tool as set forth in any one of clauses <1> to <24>, wherein a heating temperature of the base material sheet due to the opening-forming projecting mold part is the same as or greater than a glass transition temperature and is below a melting temperature of the base material sheet, and is preferably the same as or greater than a softening temperature and is below a melting temperature of the base material sheet.

<26>

A fine hollow protruding tool including a fine hollow protrusion having an opening portion,

wherein the opening portion is arranged at a position offset from a center of a tip portion of the fine hollow protrusion, and penetrates a hollow interior portion of the fine hollow protrusion; and

the fine hollow protrusion includes a rising portion rising in the shape of a convex curve toward the interior of the fine hollow protrusion, at a peripheral edge of the opening portion.

<27>

The fine hollow protruding tool as set forth in clause <26>, wherein the fine hollow protrusion has a projecting height that is preferably from 0.01 to 10 mm, and more preferably from 0.02 to 5 mm.

<28>

The fine hollow protruding tool as set forth in clause <26> or <27>, wherein the fine hollow protrusion has a tip diameter that is preferably from 1 to 500 μm, and more preferably from 5 to 300 μm.

<29>

The fine hollow protruding tool as set forth in clause <28>, wherein the opening portion has an opening area that is preferably from 0.7 to 200000 μ², and more preferably from 20 to 70000 μm².

<30>

The fine hollow protruding tool as set forth in any one of clauses <26> to <29>, wherein the fine hollow protrusion rises from a sheet-like basal member, and a basal-side opening portion is provided on a face, which is an opposite face that the fine hollow protrusion is formed, of the basal member.

<31>

The fine hollow protruding tool as set forth in clause <30>, wherein the basal-side opening portion has an opening area that is preferably from 0.007 to 20 mm², and more preferably from 0.03 to 7 μm².

<32>

The fine hollow protruding tool as set forth in any one of clauses <26> to <31>, wherein the fine hollow protruding tool is a microneedle array in which a plurality of the fine hollow protrusions are arranged on an upper face of a sheet-like basal member in such a manner that the fine hollow protrusions are aligned in each of a longitudinal direction and a lateral direction.

<33>

The fine hollow protruding tool as set forth in clause <32>, wherein a center-to-center distance in each of the longitudinal direction and the lateral direction of the fine hollow protrusions which are adjacent to each other is uniform.

<34>

The fine hollow protruding tool as set forth in clause <33>, wherein a center-to-center distance of the fine hollow protrusions which are adjacent to each other in the longitudinal direction is preferably from 0.01 to 10 mm, and more preferably from 0.05 to 5 mm.

<35>

The fine hollow protruding tool as set forth in clause <33> or <34>, wherein a center-to-center distance of the fine hollow protrusions which are adjacent to each other in the lateral direction is preferably from 0.01 to 10 mm, and more preferably from 0.05 to 5 mm.

<36>

The fine hollow protruding tool as set forth in any one of clauses <26> to <35>, wherein the opening portion is arranged at a position offset from the tip portion of the fine hollow protrusion, in a direction toward the base portion, by 2% or greater than a height of the fine hollow protrusion, preferably by 5% or greater than the height, and more preferably by 10% or greater than the height.

<37>

The fine hollow protruding tool as set forth in clause <36>, wherein the opening portion is arranged at a position offset from the base portion of the fine hollow protruding tool, in a direction toward the tip portion, by 2% or greater than the height of the fine hollow protrusion, preferably by 5% or greater than the height, and more preferably by 10% or greater than the height.

<38>

The fine hollow protruding tool as set forth in any one of clauses <26> to <36>, wherein the fine hollow protrusion has a plurality of the opening portions at positions offset from the center of the tip portion.

### EXAMPLES

Hereinafter, the present invention will be described in more detail by way of examples. However, the scope of the present invention is not limited to the following examples.

### (1) Preparation of Protrusion-Forming Projecting Mold Part 11A Included in Manufacturing Apparatus

A mold part made of SUS304, which is stainless steel, was prepared as the protrusion-forming projecting mold part 11A. The protrusion-forming projecting mold part 11A had one conical projection 110A. The projection 110A had a height (height of a tapered portion) H2 of 2.5 mm, a tip diameter D1 of 15 μm, a base diameter D2 of 0.5 mm, and a tip angle of 11 degrees.

### (2) Preparation of Opening-Forming Projecting Mold Part 11B Included in Manufacturing Apparatus

A mold part made of SUS304, which is stainless steel, was prepared as the opening-forming projecting mold part 11B. The opening-forming projecting mold part 11B had one conical projection 110B. The projection 110B had a height (height of a tapered portion) H2 of 2.5 mm, a tip diameter D1 of 15 μm, a base diameter D2 of 0.5 mm, and a tip angle of 11 degrees.

### (2) Preparation of Base Material Sheet 2A

A continuous sheet made of polylactic acid (PLA; Tg 55.8° C.) with a thickness of 0.3 mm was prepared as the base material sheet 2A.

### Example 1

A microneedle array 1M was manufactured as the fine hollow protruding tool 1 following the procedure shown in FIG. 6. Specifically, in the manufacturing apparatus 100 of this embodiment, the heating means of each of the projecting mold parts 11A and 11B was an ultrasonic vibration device.

The manufacture conditions were such that the protrusion-forming projecting mold part 11A and the opening-forming projecting mold part 11B had a frequency of ultrasonic vibrations of 20 kHz and an amplitude of ultrasonic vibrations of 40 μm. Furthermore, in the protrusion forming step, the protrusion-forming projecting mold part 11A had an insertion height of 0.7 mm, an insertion speed of 10 mm/sec, and an insertion angle θ1 of 90 degrees. Furthermore, in the opening portion forming step, the opening-forming projecting mold part 11B had an insertion amount into the fine hollow protrusions, which do not penetrate the projecting base material sheet, of 0.15 mm, an insertion speed of 30 mm/sec, an insertion angle θ2 of 270 degrees, and an offset amount from the center of the tip portion of each fine hollow protrusion, which does not penetrate the projecting base material sheet, of 10 ηm. Furthermore, the softening time was 0.1 seconds, and the cooling time was 0.5 seconds. The fine hollow protruding tool of Example 1 was manufactured following the manufacture conditions above. The temperature of the base material sheet during the insertion was 85° C., and the base material sheet had softened.

### Comparative Example 1

A fine hollow protruding tool of Comparative Example 1 was manufactured under the same manufacture conditions as in Example 1, except for an offset amount (offset amount 0 μm) from the center of the tip portion of each fine hollow protrusion which does not penetrate the projecting base material sheet.

### PERFORMANCE EVALUATION

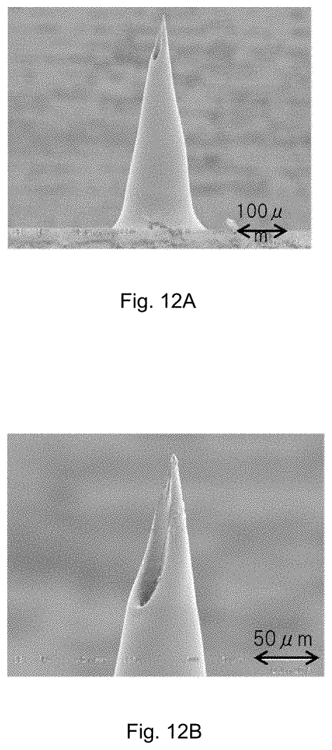

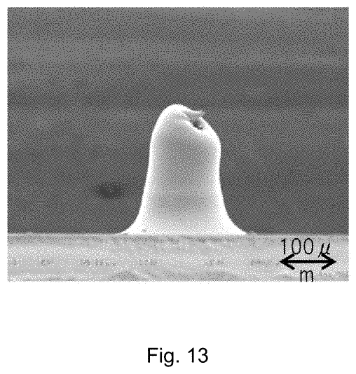

The fine hollow protruding tools of Example 1 and Comparative Example 1 were observed using a microscope, and the processing shapes of the fine hollow protrusions were evaluated based on the following criteria. Table 1 below shows the results. Photographs of the manufactured fine hollow protruding tool of Example 1 are shown in FIGS. 12A and 12B. A photograph of the manufactured fine hollow protruding tool of Comparative Example 1 is shown in FIG. 13.

TABLE 1

| | Unit | Ex. 1 | Com. Ex. 1 |
|---|---|---|---|
| Protrusion forming step | | | |
| Insertion amount | mm | 0.7 | 0.7 |
| Insertion speed | mm/s | 10 | 10 |
| Insertion angle | degrees | 90 | 90 |
| Opening portion forming step | | | |
| Insertion amount | mm | 0.15 | 0.15 |
| Insertion speed | mm/s | 30 | 30 |
| Insertion angle | degrees | 270 | 270 |
| Offset amount | μm | 10 | 0 |

As is clear from the results shown in Table 1, the fine hollow protruding tool of Example 1 had a good shape. Thus, it can be expected that, according to the manufacturing method for manufacturing the fine hollow protruding tool of Example 1, it is possible to continuously and efficiently manufacture fine hollow protruding tools that are precise in terms of the height of the fine hollow protrusions and the size of the opening portions.

Furthermore, the fine hollow protruding tool of Example 1 includes a rising portion rising toward the interior, at the peripheral edge of the opening portion, and thus the opening portion is unlikely to be crushed when piercing the skin. Thus, it can be expected that it is possible to smoothly perform piercing, and to stably deliver agents through the opening portion.

### INDUSTRIAL APPLICABILITY

According to the manufacturing method of the present invention, it is possible to manufacture a fine hollow protruding tool with a precise shape having opening portions. Furthermore, according to the fine hollow protruding tool of the present invention, it is possible to form an opening portion that is unlikely to be crushed when piercing the skin.

1. A method for manufacturing a fine hollow protruding tool, comprising:
a protrusion forming step of contacting a protrusion-forming projecting mold part including a heating means with a first face of a base material sheet containing a thermoplastic resin, and inserting the protrusion-forming projecting mold part into the base material sheet toward a second face of the base material sheet while softening, by heat, a contact portion of the base material sheet which contacts with the protrusion-forming projecting mold part to form a fine hollow protrusion which projects from the second face of the base material sheet and which does not penetrate the projecting base material sheet;
a cooling step of cooling the fine hollow protrusion in a state where the protrusion-forming projecting mold part is inserted in the fine hollow protrusion;
a release step, as a step following the cooling step, of withdrawing the protrusion-forming projecting mold part from the interior of the fine hollow protrusion to form the fine hollow protrusion having a hollow interior; and
an opening portion forming step of forming an opening portion, which penetrates an interior portion of the fine hollow protrusion, at a position offset from a center of a tip portion of the fine hollow protrusion.

2. The method for manufacturing a fine hollow protruding tool according to claim 1,
wherein the opening portion forming step is performed using an opening-forming projecting mold part including a heating means, and
in the opening portion forming step, the opening portion which penetrates the interior portion of the fine hollow protrusion is formed by contacting the opening-forming projecting mold part with the fine hollow protrusion at a position offset from the center of the tip portion, and inserting the opening-forming projecting mold part into the fine hollow protrusion while softening, by heat, a contact portion of the fine hollow protrusion which contacts with the opening-forming projecting mold part.

3. The method for manufacturing a fine hollow protruding tool according to claim 2, wherein a condition of an amount of processing heat in the protrusion forming step is different from a condition of an amount of processing heat in the opening portion forming step.

4. The method for manufacturing a fine hollow protruding tool according to claim 3, wherein a method for making the amounts of processing heat different from each other is satisfying at least one of (Condition a) to (Condition d) below:
(Condition a) an insertion speed of the protrusion-forming projecting mold part into the base material sheet and an insertion speed of the opening-forming projecting mold part into the fine hollow protrusion are such that the insertion speed in the protrusion forming step is slower than the insertion speed in the opening portion forming step;
(Condition b) in a case where the heating means of each projecting mold part is an ultrasonic vibration device, a frequency of ultrasonic waves in the protrusion-forming projecting mold part is higher than a frequency of ultrasonic waves in the opening-forming projecting mold part;
(Condition c) in a case where the heating means of each projecting mold part is an ultrasonic vibration device, an amplitude of ultrasonic waves in the protrusion-forming projecting mold part is larger than an amplitude of ultrasonic waves in the opening-forming projecting mold part; and
(Condition d) in a case where the heating means of each projecting mold part is a heater device, a heater temperature of the protrusion-forming projecting mold part is higher than a heater temperature of the opening-forming projecting mold part.

5. The method for manufacturing a fine hollow protruding tool according to claim 1, wherein the heating means is an ultrasonic vibration device.

6. The method for manufacturing a fine hollow protruding tool according to claim 2, wherein an insertion angle of the protrusion-forming projecting mold part with respect to the base material sheet in the protrusion forming step is different from an insertion angle of the opening-forming projecting mold part with respect to the base material sheet in the opening portion forming step.

7. The method for manufacturing a fine hollow protruding tool according to claim 2, wherein, in the protrusion forming step, the protrusion-forming projecting mold part is brought into contact with the first face side of the base material sheet, and, in the opening portion forming step, the opening-forming projecting mold part is brought into contact with the second face side of the base material sheet.

8. The method for manufacturing a fine hollow protruding tool according to claim 2, wherein the protrusion-forming projecting mold part is different from the opening-forming projecting mold part.

9. The method for manufacturing a fine hollow protruding tool according to claim 1, wherein, in the opening portion forming step, the opening portion is formed at a position offset from the center of the tip portion of the fine hollow protrusion, using a non-contact thermal processing means.

10. The method for manufacturing a fine hollow protruding tool according to claim 1, wherein, in the opening portion forming step, a plurality of the opening portions are formed at positions offset from the center of the tip portion of the formed fine hollow protrusion.

11. The method for manufacturing a fine hollow protruding tool according to claim 2, wherein no heating means is provided other than the heating means of the protrusion-forming projecting mold part and the opening-forming projecting mold part.

12. The method for manufacturing a fine hollow protruding tool according to claim 1, wherein a projection of the protrusion-forming projecting mold part has an outer shape that is sharper than an outer shape of the fine hollow protrusion.

13. The method for manufacturing a fine hollow protruding tool according to claim 1, wherein a projection of the protrusion-forming projecting mold part has a height that is higher than a height of the fine hollow protruding tool that is to be manufactured, and that is from 0.01 to 30 mm.

14. The method for manufacturing a fine hollow protruding tool according to claim 1, wherein a projection of the protrusion-forming projecting mold part has a tip diameter that is from 0.001 to 1 mm.

15. The method for manufacturing a fine hollow protruding tool according to claim 1, wherein a projection of the protrusion-forming projecting mold part has a base diameter that is from 0.1 to 5 mm.

16. The method for manufacturing a fine hollow protruding tool according to claim 1, wherein a projection of the protrusion-forming projecting mold part has a tip angle that is from 1 to 60 degrees.

17. The method for manufacturing a fine hollow protruding tool according to claim 1, wherein, in the protrusion forming step, a support that supports the base material sheet is provided on the second face side.

18. The method for manufacturing a fine hollow protruding tool according to claim 17, wherein an opening plate having a plurality of openings into which the projection of the protrusion-forming projecting mold part can be inserted is used as the support.

19. The method for manufacturing a fine hollow protruding tool according to claim 17, wherein, in the opening portion forming step, a support that supports the base material sheet is provided on the first face side of the base material sheet.

20. The method for manufacturing a fine hollow protruding tool according to claim 19, wherein the support provided on the first face side is an opening plate.

21. The method for manufacturing a fine hollow protruding tool according to claim 1, wherein, in the protrusion forming step, an insertion speed at which the protrusion-forming projecting mold part is inserted into the base material sheet is from 0.1 to 1000 mm/sec.

22. The method for manufacturing a fine hollow protruding tool according to claim 1, wherein, in the protrusion forming step, an insertion height by which the protrusion-forming projecting mold part is inserted into the base material sheet is from 0.01 to 10 mm.

23. The method for manufacturing a fine hollow protruding tool according to claim 1, wherein an insertion speed at which the opening-forming projecting mold part is inserted into the fine hollow protrusion which does not penetrate the projecting base material sheet is from 0.1 to 1000 mm/sec.

24. The method for manufacturing a fine hollow protruding tool according to claim 1, wherein a heating temperature of the base material sheet due to the protrusion-forming projecting mold part is the same as or greater than a glass transition temperature and is below a melting temperature of the base material sheet.

25. The method for manufacturing a fine hollow protruding tool according to claim 1, wherein a heating temperature of the base material sheet due to the protrusion-forming projecting mold part is the same as or greater than a softening temperature and is below a melting temperature of the base material sheet.

26. A fine hollow protruding tool including a fine hollow protrusion having an opening portion,
wherein the opening portion is arranged at a position offset from a center of a tip portion of the fine hollow protrusion, and penetrates a hollow interior portion of the fine hollow protrusion; and
the fine hollow protrusion includes a rising portion rising in the shape of a convex curve toward the interior of the fine hollow protrusion, at a peripheral edge of the opening portion.

27. The fine hollow protruding tool according to claim 26, wherein the fine hollow protrusion has a projecting height that is from 0.01 to 10 mm.

28. The fine hollow protruding tool according to claim 26, wherein the fine hollow protrusion has a tip diameter that is from 1 to 500 μm.

29. The fine hollow protruding tool according to claim 28, wherein the opening portion has an opening area that is from 0.7 to 200000 μm².

30. The fine hollow protruding tool according to claim 26, wherein the fine hollow protrusion rises from a sheet-like basal member, and a basal-side opening portion is provided on a face, which is an opposite face that the fine hollow protrusion is formed, of the basal member.

31. The fine hollow protruding tool according to claim 30, wherein the basal-side opening portion has an opening area that is from 0.007 to 20 mm².

32. The fine hollow protruding tool according to claim 26, wherein the fine hollow protruding tool is a microneedle array in which a plurality of the fine hollow protrusions are arranged on an upper face of a sheet-like basal member in such a manner that the fine hollow protrusions are aligned in each of a longitudinal direction and a lateral direction.

33. The fine hollow protruding tool according to claim 32, wherein a center-to-center distance in each of the longitudinal direction and the lateral direction of the fine hollow protrusions which are adjacent to each other is uniform.

34. The fine hollow protruding tool according to claim 33, wherein a center-to-center distance of the fine hollow protrusions which are adjacent to each other in the longitudinal direction is from 0.01 to 10 mm.

35. The fine hollow protruding tool according to claim 33, wherein a center-to-center distance of the fine hollow protrusions which are adjacent to each other in the lateral direction is from 0.01 to 10 mm.

36. The fine hollow protruding tool according to claim 26, wherein the opening portion is arranged at a position offset from the tip portion of the fine hollow protrusion, in a direction toward the base portion, by 2% or greater than a height of the fine hollow protrusion.

37. The fine hollow protruding tool according to claim 36, wherein the opening portion is arranged at a position offset from the base portion of the fine hollow protruding tool, in a direction toward the tip portion, by 2% or greater than the height of the fine hollow protrusion.

38. The fine hollow protruding tool according to claim 26, wherein the fine hollow protrusion has a plurality of the opening portions at positions offset from the center of the tip portion.
